SEQUENCE LISTING (19 sequences)

SEQ ID NO: 1 - DNA, 21 bp, synthetic construct
  misc_feature 1..21: note = Part of molecule containing linker and binding molecule
  source 1..21: mol_type = other DNA
ccttggcacccgagaattcca

SEQ ID NO: 2 - DNA, 48 bp, synthetic construct
  misc_feature 1..48: note = Molecule containing first ID sequence
  source 1..48: mol_type = other DNA
ttaccgacgtctactagtaaaaaaaaaaaaaaaaaaaaaaaaaaaaaa

SEQ ID NO: 3 - DNA, 48 bp, synthetic construct
  misc_feature 1..48: note = Molecule containing second ID sequence
  source 1..48: mol_type = other DNA
ttaccgacatcaggcctcaaaaaaaaaaaaaaaaaaaaaaaaaaaaaa

SEQ ID NO: 4 - RNA, 15 nt, synthetic construct
  misc_feature 1..15: note = Splint
  source 1..15: mol_type = other RNA
gtcggtaatggaatt

SEQ ID NO: 5 - DNA, 69 bp, synthetic construct
  misc_feature 1..69: note = First primer for ID sequence
  source 1..69: mol_type = other DNA
ttttttttttttttttttttttttttttttactagtagacgtcggtaatggaattctcgggtgccaagg

SEQ ID NO: 6 - DNA, 69 bp, synthetic construct
  misc_feature 1..69: note = Second primer for ID sequence
  source 1..69: mol_type = other DNA
tttttttttttttttttttttttttttttttatcgttgcgaggtcacttggaattctcgggtgccaagg

SEQ ID NO: 7 - DNA, 69 bp, synthetic construct
  misc_feature 1..69: note = Third primer for ID sequence
  source 1..69: mol_type = other DNA
ttttttttttttttttttttttttttttttcaagtatcgcgaatccgatggaattctcgggtgccaagg

SEQ ID NO: 8 - DNA, 59 bp, synthetic construct
  misc_feature 1..59: note = Part of molecule containing linker, binding molecule and IDsequence
  source 1..59: mol_type = other DNA
ccttggcacccgagaattccattaccgacaaaaaaaaaaaaaaaaaaaaaaaaaaaaaa

SEQ ID NO: 9 - DNA, 25 bp, synthetic construct
  misc_feature 1..25: note = Part of labeling sequence
  source 1..25: mol_type = other DNA
ttttttttttttttttttttttttv

SEQ ID NO: 10 - DNA, 41 bp, synthetic construct
  misc_feature 1..41: note = First binding intermediate molecule
  source 1..41: mol_type = other DNA
gtaacgatccagctgtcacttggaattctcgggtgccaagg

SEQ ID NO: 11 - DNA, 20 bp, synthetic construct
  misc_feature 1..20: note = Second binding intermediate molecule
  source 1..20: mol_type = other DNA
agtgacagctggatcgttac

SEQ ID NO: 12 - DNA, 48 bp, synthetic construct; identical to SEQ ID NO: 2
  misc_feature 1..48: note = Third molecule containig ID sequence
  source 1..48: mol_type = other DNA

SEQ ID NO: 13 - DNA, 41 bp, synthetic construct; identical to SEQ ID NO: 10
  source 1..41: mol_type = other DNA

SEQ ID NO: 14 - DNA, 59 bp, synthetic construct
  source 1..59: mol_type = other DNA
ccttggcacccgagaattccatcatttagaaaaaaaaaaaaaaaaaaaaaaaaaaaaaa

SEQ ID NO: 15 - DNA, 21 bp, synthetic construct
  source 1..21: mol_type = other DNA
ccttggcacccgagaattcca

SEQ ID NO: 16 - DNA, 30 bp, synthetic construct
  source 1..30: mol_type = other DNA
aaaaaaaaaaaaaaaaaaaaaaaaaaaaaa

SEQ ID NO: 17 - DNA, 66 bp, synthetic construct
  source 1..66: mol_type = other DNA
aatgatacggcgaccaccgagatctacacgcctgtccgcggaagcagtggtatcaacgcagagtac

SEQ ID NO: 18 - DNA, 24 bp, synthetic construct
  source 1..24: mol_type = other DNA
caagcagaagacggcatacgagat

SEQ ID NO: 19 - DNA, 33 bp, synthetic construct
  source 1..33: mol_type = other DNA
gtgactggagttccttggcacccgagaattcca

(19) United States
(12) Patent Application Publication
(10) Pub. No.: US 2024/0393342 A1
(43) Pub. Date: Nov. 28, 2024

(54) CELL LABELING MOLECULE AND METHOD FOR ANALYZING CELL

(71) Applicants: The University of Tokyo, Bunkyo-ku, Tokyo (JP); RIKEN, Wako-shi, Saitama-ken (JP)

(72) Inventors: Sadao OTA, Tokyo (JP); Ken WATANABE, Tokyo (JP); Fumiko KAWASAKI, Saitama (JP); Yuka MORI, Saitama (JP)

(73) Assignees: The University of Tokyo, Bunkyo-ku, Tokyo (JP); RIKEN, Wako-shi, Saitama-ken (JP)

(21) Appl. No.: 18/695,747

(22) PCT Filed: Sep. 27, 2022
(86) PCT No.: PCT/JP2022/036014

§ 371 Date: Jul. 2, 2024

(30) Foreign Application Priority Data
Sep. 27, 2021 (JP) 2021-157113

(51) Int. Cl. G01N 33/58; G01N 33/53

(52) U.S. Cl. CPC G01N 33/585; G01N 33/5308; G01N 33/58; G01N 33/582

(57) ABSTRACT

A cell labeling molecule comprising a particle 101 having an identifiable property, an identification sequence 102 that is identifiable and corresponds to the property of the particle 101, a cleavable linker 103 that binds the particle 101 and the identification sequence 102, and a binding molecule 104 for binding to a cell, the binding molecule being bound to the identification sequence 102.

### CROSS-REFERENCE TO RELATED APPLICATIONS

The present application is the U.S. National Stage of PCT/JP2022/036014, filed Sep. 27, 2022, which claims priority to JP 2021-157113, filed Sep. 27, 2021.

The instant application contains a Sequence Listing which has been submitted in XML format and is hereby incorporated by reference in its entirety. Said XML copy, created on Jun. 13, 2024, is named 120847-0129_sequence.xml and is 22,945 bytes.

### TECHNICAL FIELD

The present invention relates to a cell technology, and relates to a cell labeling molecule and a method for analyzing a cell.

### BACKGROUND ART

There is a proposal on a technique in which a cell and a bead are placed in each of more than one compartment, the cell and the bead in each compartment are photographed over time to monitor the dynamic change of the cell, and the compartment including the cell is identified based on the image of the identifiable bead by the compartment (e.g., see Patent Literature 1).

There is also a proposal on a technique in which a cell, a 1^{st} bead to which a 1^{st} nucleic acid is connected, and a 2^{nd} bead to which a 2^{nd} nucleic acid is connected are placed in each of more than one compartment, the cell and the 1^{st} bead in each compartment are photographed, the 1^{st} nucleic acid is cleaved from the 1^{st} bead, the cell is lysed with a cell lysis buffer placed in the compartment in advance, the 1^{st} nucleic acid and the nucleic acid contained in the cell are connected to the 2^{nd} nucleic acid, an amplified product derived from the complex of the 1^{st} nucleic acid and the 2^{nd} nucleic acid, and an amplified product derived from the complex of the nucleic acid contained in the cell and the 2^{nd} nucleic acid are produced, and the image of the cell is associated with the nucleic acid of the cell.

### CITATION LIST

### Patent Literature

Patent Literature 1: International Publication No. WO2018/203575

Patent Literature 2: International Publication No. WO2018/203576

### SUMMARY OF INVENTION

### Technical Problem

In the method described in Patent Literature 1, the nucleic acid sequence of the cell is not associated with the non-destructive information of the cell. However, the present inventors consider it beneficial to associate the non-destructive information of the cell with the nucleic acid sequence of the cell.

In the method described in Patent Literature 2, the number of cells in a compartment is one. However, when more than one cell is included in a compartment, the present inventors consider it beneficial for each of the more than one cell to associate the non-destructive information of the cell with the nucleic acid sequence of the cell. In the method described in Patent Literature 2, a cell is lysed in a compartment. For this reason, given more than one cell is placed in a compartment by the method described in Patent Literature 2, nucleic acids of the more than one cell are mixed up, thereby failing to associate the non-destructive information of the cell with nucleic acid sequence with regard to the cell of each of the more than one cell.

Further, in the methods described in Patent Literatures 1 and 2, cell is placed in a compartment. However, the present inventors consider it beneficial to associate the non-destructive information of the cell with the nucleic acid sequence in the case where cells are adherent cultured, which are considered to be difficult to place in a compartment or cells are a part of a tissue.

Under such a circumstance, it is one of the objects of the present invention to provide a cell labeling molecule capable of solving at least any of the above problems, and a method for analyzing a cell.

### Solution to Problem

A cell labeling molecule according to the 1^{st} aspect of the present invention comprises a particle having an identifiable property, an identification sequence that is identifiable and corresponds to the property of the particle, a cleavable linker that binds the particle and the identification sequence, and a binding molecule for binding to a cell, the binding molecule being bound to the identification sequence.

In the cell labeling molecule according to the 1^{st} aspect, the property of the particle bound to the identification sequence may be capable of being specified based on the identification sequence.

In the cell labeling molecule according to the 1^{st} aspect, the particle may be a bead.

In the cell labeling molecule according to the 1^{st} aspect, the identification sequence may be a nucleic acid, or an analog thereof.

In the cell labeling molecule according to the 1^{st} aspect, the identification sequence may be a deoxyribonucleic acid, or an analog thereof.

In the cell labeling molecule according to the 1^{st} aspect, the binding molecule may be a molecule capable of binding to a molecule of a cell. The binding molecule may be a molecule capable of binding to a molecule of a cell by a covalent bond. The binding molecule may be at least one selected from the group consisting of nucleic acids, analogs of nucleic acids, peptides, analogs of peptides, proteins, lipids, sugars, and analogs of sugars.

The cell labeling molecule according to the 1^{st} aspect may further comprise a sequence labeling molecule bound to the identification sequence and/or the binding molecule.

In the cell labeling molecule according to the 1^{st} aspect, the sequence labeling molecule may include a fluorescent molecule.

In the cell labeling molecule according to the 1^{st} aspect, the sequence labeling molecule may include an affinity tag.

A kit according to the 2^{nd} aspect of the present invention comprises more than one cell labeling molecule, wherein each of the more than one cell labeling molecule comprises a particle having an identifiable property, an identification sequence that is identifiable and corresponds to the property of the particle, a cleavable linker that binds the particle and the identification sequence, and a binding molecule for binding to a cell, the binding molecule being bound to the identification sequence, wherein at least part of the more than one cell labeling molecule has different properties from each other.

In each of the more than one cell labeling molecule comprised in the kit according to the 2^{nd} aspect, the property of the particle to which the identification sequence is bound may be capable of being specified based on the identification sequence.

In each of the more than one cell labeling molecule according to the 2^{nd} aspect, the particle may be a bead.

In each of the more than one cell labeling molecule according to the 2^{nd} aspect, the identification sequence may be a nucleic acid or an analog thereof.

In each of the more than one cell labeling molecule comprised in the kit according to the 2^{nd} aspect, the identification sequence may be a deoxyribonucleic acid or an analog thereof.

In each of the more than one cell labeling molecule comprised in the kit according to the 2^{nd} aspect, the binding molecule may be a molecule capable of binding to a molecule of a cell. The binding molecule may be at least one selected from the group consisting of nucleic acids, analogs of nucleic acids, peptides, analogs of peptides, proteins, lipids, sugars, and analogs of sugars.

The kit according to the 2^{nd} aspect may further comprise a binding mediating molecule that mediates the binding of the binding molecule to a cell.

In the kit according to the 2^{nd} aspect, the binding mediating molecule may be a molecule capable of binding to the binding molecule.

In the kit according to the 2^{nd} aspect, the binding mediating molecule may be a molecule capable of binding to a molecule of a cell. The binding mediating molecule may be a molecule capable of binding to a molecule of a cell by a covalent bond. The binding mediating molecule may be at least one selected from the group consisting of nucleic acids, analogs of nucleic acids, peptides, analogs of peptides, proteins, lipids, sugars, and analogs of sugars.

Each of more than one cell labeling molecule comprised in the kit according to the 2^{nd} aspect may further comprise a sequence labeling molecule bound to the identification sequence and/or the binding molecule.

In each of the more than one cell labeling molecules comprised in the kit according to the 2^{nd} aspect, the sequence labeling molecule may include a fluorescent molecule.

In each of the more than one cell labeling molecule comprised in the kit according to the 2^{nd} aspect, the sequence labeling molecule may include an affinity tag.

The method for analyzing a cell according to the 3^{rd} aspect of the present invention comprises (a) adding, to at least one cell, a cell labeling molecule comprising a particle having an identifiable property, an identification sequence that is identifiable and corresponds to the property of the particle, a cleavable linker that binds the particle and the identification sequence, and a binding molecule for binding to a cell, the binding molecule being bound to the identification sequence, (b) obtaining the property of the particle, and non-destructive information of the at least one cell, (c) cleaving the linker and binding the identification sequence to the at least one cell through the binding molecule, (d) isolating the at least one cell to which the identification sequence is bound, to read out the identification sequence and the nucleic acid sequence of the at least one cell, and (e) associating the non-destructive information of the at least one cell with the nucleic acid sequence of the at least one cell.

In the method for analyzing a cell according to the 3^{rd} aspect, the at least one cell is more than one cell, each of the more than one cell to which the identification sequence is bound is isolated, the identification sequence and the nucleic acid sequence of the cell are read out in each isolated cell, and with regard to each of the more than one cell, the non-destructive information of the cell may be associated with the nucleic acid sequence of the cell.

In the method for analyzing a cell according to the 3^{rd} aspect, the property of the particle and the non-destructive information of the at least one cell may be optically obtained.

In the method for analyzing a cell according to the 3^{rd} aspect, the binding molecule may bind to the cell through the binding mediating molecule.

The method for analyzing a cell according to the 3^{rd} aspect may further comprise introducing the binding mediating molecule to a cell.

In the method for analyzing a cell according to the 3^{rd} aspect, in the reading out the identification sequence and the nucleic acid sequence of the at least one cell, the identification sequence and the nucleic acid sequence of the at least one cell may be read out with respect to each single cell.

In the method for analyzing a cell according to the 3^{rd} aspect, the property of the particle and the non-destructive information of the at least one cell may be obtained using the same device. The device may be an optical device.

In the method for analyzing a cell according to the 3^{rd} aspect, the property of the particle and the non-destructive information of the at least one cell may be obtained by the same method. The method may be an optical method.

In the method for analyzing a cell according to the 3^{rd} aspect, the property of the particle and the non-destructive information of the at least one cell may be obtained simultaneously.

In the method for analyzing a cell according to the 3^{rd} aspect, data including the property of the particle and the non-destructive information of the at least one cell may be obtained. The data may be image data.

In the method for analyzing a cell according to the 3^{rd} aspect, the non-destructive information of the at least one cell may include information on morphology of the at least one cell.

In the method for analyzing a cell according to the 3^{rd} aspect, the at least one cell may be in a compartment.

In the method for analyzing a cell according to the 3^{rd} aspect, in the adding the cell labeling molecule to the at least one cell, the cell labeling molecule may be placed in the compartment.

In the method for analyzing a cell according to the 3^{rd} aspect, the compartment may be a gel. The at least one cell may be present in the gel. The compartment may be a gel that has an internal space. The compartment may include a liquid in the internal space. The at least one cell may be present in the liquid. The liquid may be a culture solution. The compartment may be in an oil. The compartment may be in an aqueous solution. The compartment may be a droplet. The at least one cell may be present in the droplet. The droplet may include a gel. The at least one cell may be present in the gel in the droplet. The droplet may be aqueous. The droplet may be in an oil. The droplet may be covered with an oil membrane. The droplet covered with the oil membrane may be in an aqueous solution.

The method for analyzing a cell according to the 3^{rd} aspect may further comprise isolating the at least one cell from the compartment.

In the method for analyzing a cell according to the 3^{rd} aspect, the at least one cell may be isolated by flow cytometry.

In the method for analyzing a cell according to the 3^{rd} aspect, the at least one cell may be isolated using an affinity tag.

In the method for analyzing a cell according to the 3^{rd} aspect, the cell labeling molecule further comprises a sequence labeling molecule bound to the identification sequence and/or the binding molecule, and in the isolation, the sequence labeling molecule may be used.

The method for analyzing a cell according to the 4^{th} aspect comprises (a) adding, to at least one cell, more than one cell labeling molecule respectively comprising a particle having an identifiable property, an identification sequence that is identifiable and corresponds to the property of the particle, a cleavable linker that binds the particle and the identification sequence, and a binding molecule for binding to a cell, the binding molecule being bound to the identification sequence, wherein at least part of the more than one cell labeling molecule has different properties of the particles from each other, (b) obtaining the properties of the more than one particle, and the non-destructive information of the at least one cell, (c) cleaving more than one linker to bind more than one identification sequence to the at least one cell through more than one binding molecule, (d) isolating the at least one cell to which more than one identification sequence is bound, to read out the more than one identification sequence and a nucleic acid sequences of the at least one cell, and (e) associating the non-destructive information of the at least one cell with the nucleic acid sequence of the at least one cell.

In the method for analyzing a cell according to the 4^{th} aspect, the at least one cell is more than one cell, each of the more than one cell to which the identification sequence is bound is isolated, the identification sequence and the nucleic acid sequence of the cell are read out in each isolated cell, and with regard to each of the more than one cell, the non-destructive information of the cell may be associated with the nucleic acid sequence of the cell.

In the method for analyzing a cell according to the 4^{th} aspect, the properties of the more than one particle and the non-destructive information of the at least one cell may be optically obtained.

In the method for analyzing a cell according to the 4^{th} aspect, the binding molecule may bind to the cell through the binding mediating molecule.

The method for analyzing a cell according to the 4^{th} aspect may further comprise introducing the binding mediating molecule to a cell.

In the method for analyzing a cell according to the 4^{th} aspect, in the reading out the more than one identification sequence and the nucleic acid sequence of the at least one cell, the more than one identification sequence and the nucleic acid sequence of the cell may be read out with respect to each single cell.

In the method for analyzing a cell according to the 4^{th} aspect, the properties of the more than one particle and the non-destructive information of the at least one cell may be obtained using the same device. The device may be an optical device.

In the method for analyzing a cell according to the 4^{th} aspect, the properties of the more than one particle and the non-destructive information of the at least one cell may be obtained by the same method. The method may be an optical method.

In the method for analyzing a cell according to the 4^{th} aspect, the properties of the more than one particle and the non-destructive information of the at least one cell may be obtained simultaneously.

In the method for analyzing a cell according to the 4^{th} aspect, data including the properties of the more than one particle and the non-destructive information of the at least one cell may be obtained. The data may be image data.

In the method for analyzing a cell according to the 4^{th} aspect, the non-destructive information of the at least one cell may include information on morphology of the at least one cell.

In the method for analyzing a cell according to the 4^{th} aspect, the at least one cell may be placed in a compartment.

In the method for analyzing a cell according to the 4^{th} aspect, in the addition of the more than one cell labeling molecule to the at least one cell, the more than one cell labeling molecule may be placed in a compartment.

In the method for analyzing a cell according to the 4^{th} aspect, the compartment may be a gel. The at least one cell may be present in the gel. The compartment may be a gel that has an internal space. The compartment may include a liquid in the internal space. The at least one cell may be present in the liquid. The liquid may be a culture solution. The compartment may be in an oil. The compartment may be in an aqueous solution. The compartment may be a droplet. The at least one cell may be present in the droplet. The droplet may include a gel. The at least one cell may be present in the gel in the droplet. The droplet may be aqueous. The droplet may be in an oil. The droplet may be covered with an oil membrane. The droplet covered with the oil membrane may be in an aqueous solution.

The method for analyzing a cell according to the 4^{th} aspect may further comprise isolating the at least one cell from the compartment.

In the method for analyzing a cell according to the 4^{th} aspect, the at least one cell may be isolated by flow cytometry.

In the method for analyzing a cell according to the 4^{th} aspect, the at least one cell may be isolated using an affinity tag.

In the method for analyzing a cell according to the 4^{th} aspect, each of the more than one cell labeling molecule may further comprise a sequence labeling molecule bound to the identification sequence and/or the binding molecule, and in the isolation, the sequence labeling molecule may be used.

The method for analyzing a cell according to the 5^{th} aspect comprises (a) binding, to at least one cell, a cell labeling molecule comprising a particle having an identifiable property, an identification sequence that is identifiable and corresponds to the property of the particle, a cleavable linker that binds the particle and the identification sequence, and a binding molecule for binding to a cell, the binding molecule being bound to the identification sequence, (b) obtaining the property of the particle, and non-destructive information of the at least one cell, (c) cleaving the linker to release the particle from the cell, (d) isolating the at least one cell to which the identification sequence is bound, to read out the identification sequence and the nucleic acid sequence of the at least one cell, and (e) associating the non-destructive information of the at least one cell with the nucleic acid sequence of the at least one cell.

In the method for analyzing a cell according to the 5^{th} aspect, the at least one cell is more than one cell, each of the more than one cell to which the identification sequence is bound is isolated, the identification sequence and the nucleic acid sequence of the cell are read out in each isolated cell, and with regard to each of the more than one cell, the non-destructive information of the cell may be associated with the nucleic acid sequence of the cell.

In the method for analyzing a cell according to the 5^{th} aspect, the property of the particle and the non-destructive information of the at least one cell may be optically obtained.

In the method for analyzing a cell according to the 5^{th} aspect, the binding molecule may bind to the cell through the binding mediating molecule.

The method for analyzing a cell according to the 5^{th} aspect may further comprise introducing the binding mediating molecule to a cell.

In the method for analyzing a cell according to the 5^{th} aspect, in the reading out the identification sequence and the nucleic acid sequence of the at least one cell, the identification sequence and the nucleic acid sequence of the cell may be read out with respect to each single cell.

In the method for analyzing a cell according to the 5^{th} aspect, the property of the particle and the non-destructive information of the at least one cell may be obtained using the same device. The device may be an optical device.

In the method for analyzing a cell according to the 5^{th} aspect, the property of the particle and the non-destructive information of the at least one cell may be obtained by the same method. The method may be an optical method.

In the method for analyzing a cell according to the 5^{th} aspect, the property of the particle and the non-destructive information of the at least one cell may be obtained simultaneously.

In the method for analyzing a cell according to the 5^{th} aspect, data including the property of the particle and the non-destructive information of the at least one cell may be obtained. The data may be image data.

In the method for analyzing a cell according to the 5^{th} aspect, the non-destructive information of the at least one cell may include information on morphology of the at least one cell.

In the method for analyzing a cell according to the 5^{th} aspect, when the cell labeling molecule is bound to the at least one cell, the at least one cell may be adherent cultured.

In the method for analyzing a cell according to the 5^{th} aspect, before isolating the at least one cell, the at least one cell may be detached from an incubator.

In the method for analyzing a cell according to the 5^{th} aspect, when the cell labeling molecule is bound to the at least one cell, the at least one cell may be a part of a tissue.

In the method for analyzing a cell according to the 5^{th} aspect, before isolating the at least one cell, the at least one cell may be dissociated from the tissue.

In the method for analyzing a cell according to the 5^{th} aspect, the at least one cell may be isolated by flow cytometry.

In the method for analyzing a cell according to the 5^{th} aspect, the at least one cell may be isolated using an affinity tag.

In the method for analyzing a cell according to the 5^{th} aspect, the cell labeling molecule further comprises a sequence labeling molecule bound to the identification sequence and/or the binding molecule, and in the isolation, the sequence labeling molecule may be used.

The method for analyzing a cell according to the 6^{th} aspect of the present invention comprises (a) binding, to at least one cell, more than one cell labeling molecule respectively comprising a particle having an identifiable property, an identification sequence that is identifiable and corresponds to the property of the particle, a cleavable linker that binds the particle and the identification sequence, and a binding molecule for binding to a cell, the binding molecule being bound to the identification sequence, wherein at least part of the more than one cell labeling molecule has different properties of the particles from each other, (b) obtaining the properties of more than one particle, and the non-destructive information of the at least one cell, (c) cleaving the linker, to release more than one particle from the cell, (d) isolating the at least one cell to which more than one identification sequence is bound, to read out the more than one identification sequence and a nucleic acid sequence of the at least one cell, and (e) associating the non-destructive information of the at least one cell with the nucleic acid sequence of the at least one cell.

In the method for analyzing a cell according to the 6^{th} aspect, the at least one cell is more than one cell, each of the more than one cell to which the identification sequence is bound is isolated, the identification sequence and the nucleic acid sequence of the cell are read out in each isolated cell, and with regard to each of the more than one cell, the non-destructive information of the cell may be associated with the nucleic acid sequence of the cell c.

In the method for analyzing a cell according to the 6^{th} aspect, the property of the particle and the non-destructive information of the at least one cell may be optically obtained.

In the method for analyzing a cell according to the 6^{th} aspect, the binding molecule may bind to the cell through the binding mediating molecule.

The method for analyzing a cell according to the 6^{th} aspect may further comprise introducing the binding mediating molecule to the cell.

In the method for analyzing a cell according to the 6^{th} aspect, in the reading out the more than one identification sequence and the nucleic acid sequence of the at least one cell, the more than one identification sequence and the nucleic acid sequence of the cell may be read out with respect to each single cell.

In the method for analyzing a cell according to the 6^{th} aspect, the properties of the more than one particle and the non-destructive information of the at least one cell may be obtained using the same device. The device may be an optical device.

In the method for analyzing a cell according to the 6^{th} aspect, the properties of the more than one particle and the non-destructive information of the at least one cell may be obtained by the same method. The method may be an optical method.

In the method for analyzing a cell according to the 6^{th} aspect, the properties of the more than one particle and the non-destructive information of the at least one cell may be obtained simultaneously.

In the method for analyzing a cell according to the 6^{th} aspect, data including the property of the more than one particle and the non-destructive information of the at least one cell may be obtained. The data may be image data.

In the method for analyzing a cell according to the 6^{th} aspect, the non-destructive information of the at least one cell may include information on morphology of the at least one cell.

In the method for analyzing a cell according to the 6^{th} aspect, when the more than one cell labeling molecule is bound to the at least one cell, the at least one cell may be adherent cultured.

In the method for analyzing a cell according to the 6^{th} aspect, before isolating the at least one cell, the at least one cell may be detached from an incubator.

In the method for analyzing a cell according to the 6^{th} aspect, when the more than one cell labeling molecule is bound to the at least one cell, the at least one cell may be a part of a tissue.

In the method for analyzing a cell according to the 6^{th} aspect, before isolating the at least one cell, the at least one cell may be dissociated from the tissue.

In the method for analyzing a cell according to the 6^{th} aspect, the at least one cell may be isolated by flow cytometry.

In the method for analyzing a cell according to the 6^{th} aspect, the at least one cell may be isolated using an affinity tag.

In the method for analyzing a cell according to the 6^{th} aspect, each of the more than one cell labeling molecule may further comprise a sequence labeling molecule bound to the identification sequence and/or the binding molecule, and in the isolation, the sequence labeling molecule may be used.

### Advantageous Effects of Invention

According to the present invention, it is possible to provide a cell labeling molecule capable of efficiently analyzing a cell, and a method for analyzing a cell.

### BRIEF DESCRIPTION OF DRAWINGS

FIG. 1 schematically shows a cell labeling molecule according to an embodiment.

FIG. 2 schematically shows a cell labeling molecule according to an embodiment.

FIG. 3 schematically shows a cell labeling molecule according to an embodiment.

FIG. 4 schematically shows a cell labeling molecule according to an embodiment.

FIG. 5 schematically shows a cell labeling molecule according to an embodiment.

FIG. 6 schematically shows a cell labeling molecule according to an embodiment.

FIG. 7 schematically shows a cell labeling molecule according to an embodiment.

FIG. 8 schematically shows a cell labeling molecule according to an embodiment.

FIG. 9 is a flow chart showing a method for analyzing a cell according to an embodiment.

FIG. 10 schematically shows a compartment according to an embodiment.

FIG. 11 schematically shows a compartment according to an embodiment.

FIG. 12 is a flow chart showing a method for analyzing a cell according to an embodiment.

FIG. 13 is a schematic drawing showing a method for analyzing a cell according to an embodiment.

FIG. 14 schematically shows an acrylamide bead to which a linker and a binding molecule-containing molecule are bound according to Example 1 of an embodiment.

FIG. 15 schematically shows an acrylamide bead to which a linker, a binding molecule-containing molecule, and an identification sequence-containing molecule are bound according to Example 2 of an embodiment.

FIG. 16 schematically shows a microfluidic chip according to Example 8 of an embodiment.

FIGS. 17A-17B show photographs of compartments according to Example 9 of an embodiment.

FIGS. 18A-18B show photographs of compartment a according to Example 9 of an embodiment.

FIGS. 19A-19B show photographs of compartment a according to Example 9 of an embodiment.

FIG. 20 shows a photograph of cells and compartment forming solution according to Example 10 of an embodiment.

FIG. 21 shows a photograph of compartments according to Example 10 of an embodiment.

FIG. 22 schematically shows a flow channel according to Example 10 of an embodiment.

FIGS. 23A-23B show photographs of compartments according to Example 10 of an embodiment.

FIGS. 24A-24B show photographs of compartments according to Reference Example 1 of an embodiment.

FIG. 25 shows a photograph of light pattern of a UV light according to Reference Example 1 of an embodiment.

FIG. 26 shows a photograph of compartments according to Reference Example 1 of an embodiment.

FIGS. 27A-27B show photographs of compartments according to Reference Example 2 of an embodiment.

FIG. 28 shows a photograph of compartments according to Reference Example 2 of an embodiment.

FIG. 29 schematically shows an acrylamide bead to which a linker and a binding molecule-containing molecule, and a tert-butoxycarbonyl group are bound according to Example 12 of an embodiment.

FIG. 30 schematically shows an acrylamide bead to which a linker and a binding molecule-containing molecule according to Example 12 of an embodiment, and a fluorescent dye are bound.

FIG. 31 schematically shows an acrylamide bead to which a linker, a binding molecule-containing molecule, and an identification sequence-containing molecule according to Example 12 of an embodiment, and a fluorescent dye are bound.

FIG. 32 shows fluorescence micrographs of cells according to Example 12 of an embodiment.

FIG. 33 shows graphs demonstrating fluorescence intensity with respect to each solution according to Example 12 of an embodiment.

FIG. 34 shows a graph demonstrating detection intensity of the 1^{st} identification sequence or the 2^{nd} identification sequence according to Example 13 of an embodiment.

FIGS. 35A-35C show dot plots obtained by FACS according to Example 14 of an embodiment.

FIG. 36 shows a graph demonstrating total signal count with respect to each magnetic bead according to Example 15 of an embodiment.

FIG. 37 shows a graph demonstrating a signal pattern with respect to each cell according to Example 15 of an embodiment.

FIGS. 38A-38C show graphs demonstrating clustered data according to Example 15 of an embodiment.

### DESCRIPTION OF EMBODIMENTS

Hereinafter, embodiments of the present invention will be described in detail. The following embodiments are given to illustrate materials, substances, chemicals, and methods for embodying the technical ideas of this invention, and the technical ideas of this invention do not intend to limit the combinations of element members and the like to the following descriptions. Various alterations can be added to the technical ideas of this invention within the scope of the patent claims.

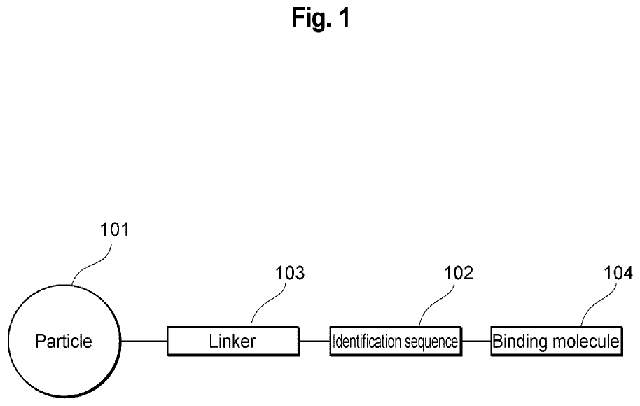

A cell labeling molecule according to an embodiment, as schematically shown in FIG. 1, comprises a particle 101 having an optically identifiable property, an identification sequence 102 that is identifiable and corresponds to the property of the particle 101, a cleavable linker 103 that binds the particle 101 and the identification sequence 102, and a binding molecule 104 for binding to a cell, the binding molecule being bound to the identification sequence 102. In the present disclosure, the binding also includes non-covalent bonds such as hydrophobic interaction.

The particle 101 is, for example, a bead. Examples of the material for the particle 101 are not limited, and include semiconductors such as cadmium selenide (CdSe), zinc sulfide (ZnS), cadmium sulfide (CdS), zinc selenide (ZnSe), and zinc oxide (ZnO), and analogs thereof; metals such as gold, silver, and platinum, and analogs thereof; hydrogels such as acrylamide, agarose, collagen, alginic acid, hyaluronic acid, chitosan, gelatin, Poly(ethylene glycol) diacrylate (PEGDA), and PEG, and analogs thereof; and resins such as polystyrene, polypropylene, and hydrophilic vinyl polymers, and analogs thereof. Additionally, the material for the particle 101 can be copolymers or mixtures thereof.

Examples of the optically identifiable property of the particle 101 are not limited, and include the particle size of the particle 101, the shape of the particle 101, the color of transmitted light of the particle 101, the wavelength of transmitted light of the particle 101, the transmitted light spectrum of the particle 101, the phase shift of transmitted light of the particle 101, the transmittance of the particle 101, the absorption spectrum of the particle 101, the absorptivity of the particle 101, the color of reflected light of the particle 101, the wavelength of reflected light of the particle 101, the reflected light spectrum of the particle 101, the phase shift of reflected light of the particle 101, the reflectance of the particle 101, the color of scattered light of the particle 101, the wavelength of scattered light of the particle 101, the scattered light spectrum of the particle 101, the phase shift of scattered light of the particle 101, the color of fluorescence emitted from the particle 101, the wavelength of fluorescence emitted from the particle 101, and the spectrum of fluorescence emitted from the particle 101. The wavelength bands of transmitted light, absorption, reflected light, and scattered light are not particularly limited, and can be a visible light or an infrared light. The scattered light may include the Raman scattered light. For example, the optically identifiable property can be imparted to the particle 101 by adjusting at least any of the material and the production method of the particle 101.

When a plurality of cell labeling molecules are used, the properties of the plurality of particles 101 of the plurality of cell labeling molecules may be different from each other so that the plurality of cell labeling molecules can be identifiable to each other. For example, when there are three particle sizes, three colors of the reflected lights, and six reflectances of reflected lights, 50 or more combinations of the particle sizes, reflected lights, and reflectances can be made. Accordingly, the combinations of three particle sizes, three colors of the reflected lights, and six reflectances of reflected lights can produce the particles 101 having 50 or more optically identifiable properties to each other.

The identification sequence 102 comprises, for example, a nucleic acid or an analog thereof, but not limited thereto. Examples of the nucleic acid are not limited, but include deoxyribonucleic acids, ribonucleic acids, and artificial nucleic acids. The identification sequence 102 includes more than one base sequence. The base includes at least any of adenine (A), guanine (G), cytosine (C), thymine (T), and uracil (U).

The identification sequence 102 has an identifiable sequence corresponded to the property of the particle. In a single cell labeling molecule, the identification sequence 102 and the property of the particle 101 are in a unique relationship. Accordingly, when the identification sequence 102 of the single cell labeling molecule is identified, the property of the particle 101 that this single cell labeling molecule has or had can be identified. Conversely, when the property of the particle 101 of a single cell labeling molecule is identified, the identification sequence 102 that this single cell labeling molecule has or had can be identified.

When the plurality of cell labeling molecules are used, in each of the plurality of cell labeling molecules, the identification sequence 102 and the property of the particle 101 are in a unique relationship. The plurality of identification sequences 102 of the plurality of cell labeling molecules may be different from each other so that the plurality of cell labeling molecules are identifiable to each other.

The base length of the identification sequence 102 is not particularly limited, but is 5 or more and 120 or less, 5 or more and 80 or less, 5 or more and 50 or less, or 10 or more and 40 or less. For example, there are 4 types of bases, and when the base length of the sequence is 12, 10000000 or more sequence combinations can be made.

The identification sequence is also referred to as a barcode sequence.

The linker 103 that binds the particle 101 and the identification sequence 102 can be cleaved in any method. The linker 103 comprises, for example, a molecule that is cleavable by at least any of light irradiation, chemical reaction, and enzyme reaction, but not limited thereto. The light irradiation includes UV irradiation. The chemically cleavable molecule includes, for example, a disulfide bond. Additionally, the linker 103 comprises, for example, a molecule that can be cleaved in at least any of temperature- and pH-dependent manners, but not limited thereto.

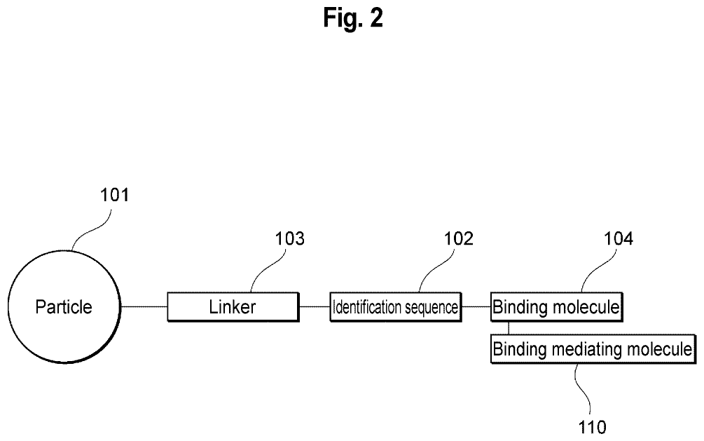

The binding molecule 104 is not limited and, for example, can be a molecule capable of binding to a molecule of a cell. The binding molecule can be a molecule capable of binding to a molecule of a cell by a covalent bond. The binding molecule can comprise at least any of the cell-bindable nucleic acids, analogs of nucleic acids, peptides, analogs of peptides, proteins, lipids, sugars, analogs of sugars, and compounds. Examples of the protein include ligands and antibodies. Examples of the lipid include cholesterol. The binding molecule 104 does not necessarily bind directly to a cell. For example, as shown in FIG. 2, the binding molecule 104 can also bind to a cell through a binding mediating molecule 110 bound to the cell. The binding mediating molecule 110 can be bound to a cell in advance. In this instance, the binding molecule 104 of the cell labeling molecule binds to the binding mediating molecule bound to a cell. The binding molecule 104 and the binding mediating molecule 110 can comprise sequences that are complementary to each other. The binding molecule 104 and the binding mediating molecule 110 can also comprise proteins that bind to each other. Alternatively, both of the binding molecule 104 and the binding mediating molecule 110 are capable of binding to a cell, and the binding mediating molecule 110 can reinforce the binding force between the binding molecule 104 and the cell.

A cell can be included in a compartment. The compartment is not limited, and examples include droplets and gel particles. The compartment can be in an oil. The compartment can be in an aqueous solution. A cell can be present in a droplet. The droplet may include a gel. A cell can be present in the gel in the droplet. The droplet can be aqueous. The aqueous droplet can be in an oil. The droplet can be covered with an oil membrane. The droplet covered with the oil membrane can be in an aqueous solution. A cell can be present in a gel particle. The gel particle may include an internal space. The gel particle may include a liquid in the internal space. A cell can be present in the liquid in the internal gel particle. The liquid can be a culture solution.

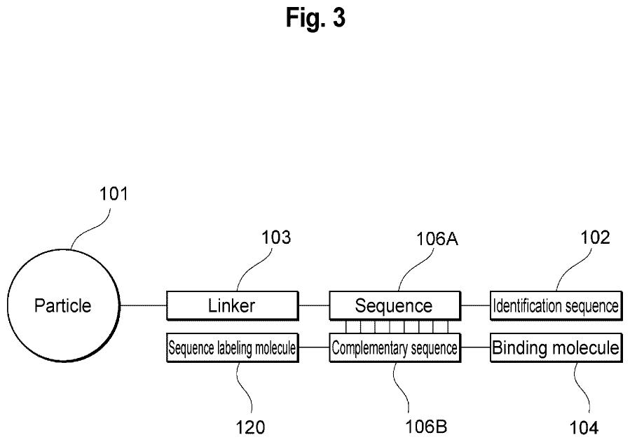

As shown in FIG. 3, the cell labeling molecule may further comprise a sequence labeling molecule 120 bound to the identification sequence 102 and/or the binding molecule 104. The sequence labeling molecule 120 is bound to the identification sequence 102 and/or binding molecule 104 even after the linker 103 is cleaved. The sequence labeling molecule 120 can also be connected through a complementary sequence 106B that is complementary to a sequence 106A between the linker 103 and the identification sequence 102. The sequence labeling molecule 120 can also include a fluorescent molecule. The sequence labeling molecule 120 may also include a magnetic substance. The sequence labeling molecule 120 may also include an affinity tag. The affinity tag may also comprise at least any of nucleic acids, analogs of nucleic acids, peptides, analogs of peptides, proteins, small molecules, lipids, sugars, analogs of sugars, and compounds. Examples of the small molecule include biotin. Examples of the protein include avidin, antibodies, and antigens. It is desirable that the affinity tag does not bind to a binding target of the binding molecule.

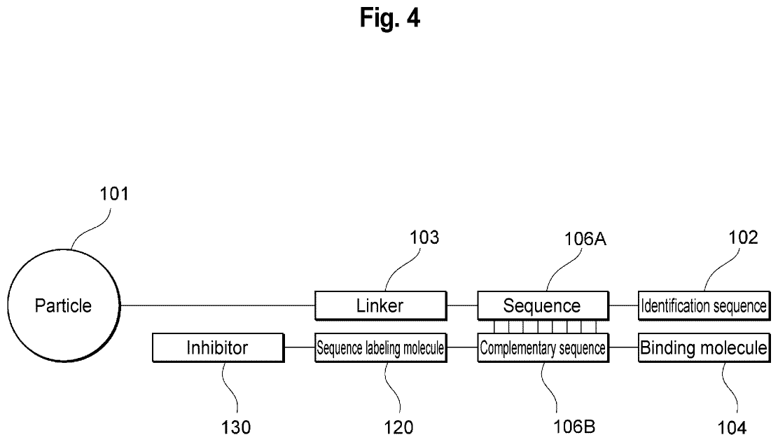

As shown in FIG. 4, the cell labeling molecule may further comprise an inhibitor 130 that inhibits the function of the sequence labeling molecule 120, and the inhibitory function deactivates under the same conditions that cleave the linker 103. When the cell labeling molecule comprises the inhibitor 130, the function of the sequence labeling molecule 120 is inhibited. When the linker 103 of the cell labeling molecule is cleaved, the inhibitory function of the inhibitor 130 deactivates, whereby the function of the sequence labeling molecule 120 is not inhibited, but demonstrated. The inhibitor 130 can be cleaved from the cell labeling molecule under the conditions that cleave the linker 103 of the cell labeling molecule. The inhibitor 130 is bound to, for example, the sequence labeling molecule 120. When the sequence labeling molecule 120 includes a fluorescent molecule, the inhibitor 130 is a fluorescent inhibitor. When the sequence labeling molecule 120 includes a magnetic substance, the inhibitor 130 is a magnetic inhibitor. When the sequence labeling molecule 120 includes a nucleic acid, the inhibitor 130 is a base-paring inhibitor. For example, a base to which 6-nitropiperonyloxymethyl is bound is incapable of forming base pairs, but when 6-nitropiperonyloxymethyl is removed by UV irradiation, the base can form base pairs.

The cell labeling molecule may further comprise, for example, a priming site complementary to a PCR primer. The priming site can be connected or inserted to at least any of the linker 103, the identification sequence 102, and the binding molecule 104 as long as it remains bound to the identification sequence 102 even when the linker 103 is cleaved.

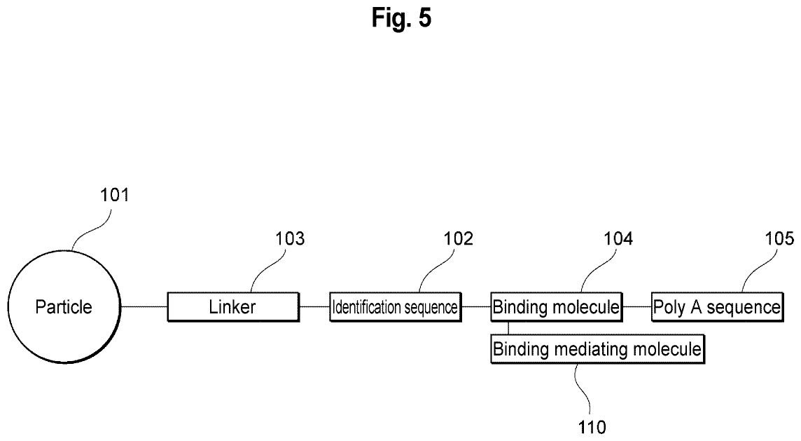

The cell labeling molecule may further comprise other molecules. The other molecules can be connected or inserted to at least any of the linker 103, the identification sequence 102, and the binding molecule 104. For example, as shown in FIG. 5, the cell labeling molecule may further comprise a poly A sequence 105. For example, using a poly T sequence complementary to the poly A sequence 105, a sequence recognizing molecule such as a fluorescent molecule can be bound to the cell labeling molecule.

When the linker 103 is cleaved, the order of the linker 103, the identification sequence 102, and the binding molecule 104 in the cell labeling molecule are not limited as long as the identification sequence 102 and the binding molecule 104 can be released from the particle 101. The linker 103, identification sequence 102, and the binding molecule 104 can be connected in series, or branched connected.

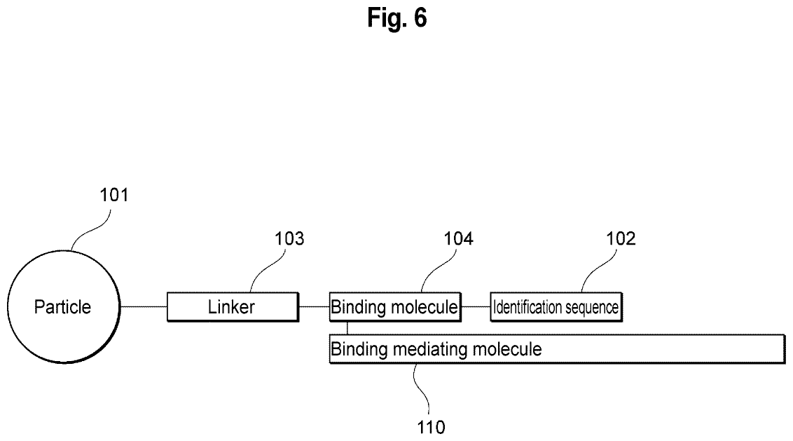

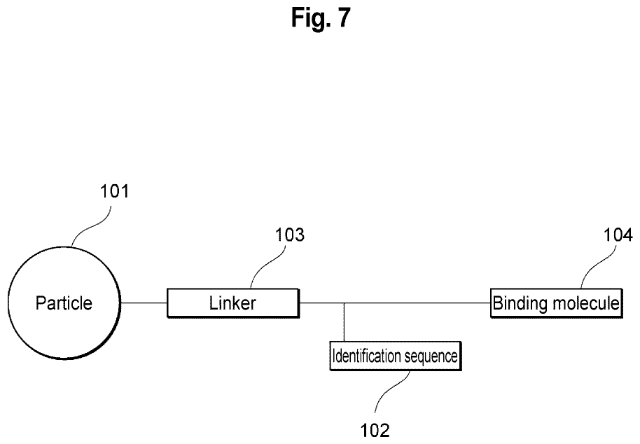

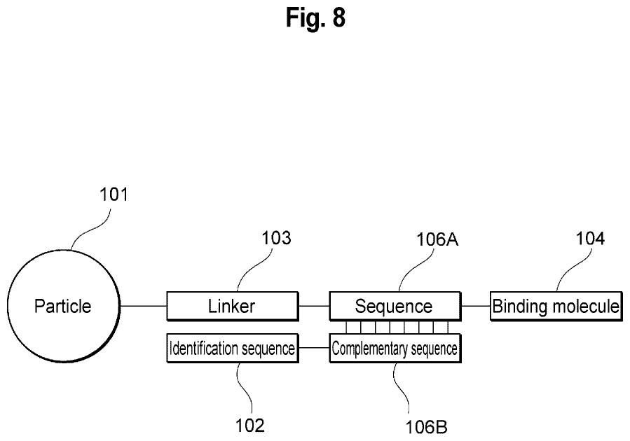

For example, as shown in FIG. 6, the binding molecule 104 can be disposed between the linker 103 and the identification sequence 102. As shown in FIG. 7, the identification sequence 102 can be branched connected between the linker 103 and the binding molecule 104. As shown in FIG. 8, the identification sequence 102 can also be connected through a complementary sequence 106B complementary to the sequence 106A between the linker 103 and the binding molecule 104.

To a single particle 101, a plurality of linkers 103, a plurality of identification sequences 102, and a plurality of binding molecules 104 may be connected. It should be noted that the plurality of identification sequences 102 to be connected to the single particle 101 are all the same.

A kit according to the embodiment comprises a plurality of cell labeling molecules. Each of the plurality of cell labeling molecules is as described above. The properties of the particles in the plurality of cell labeling molecules are different from each other. Accordingly, the plurality of one cell labeling molecule can be identifiable to each other.

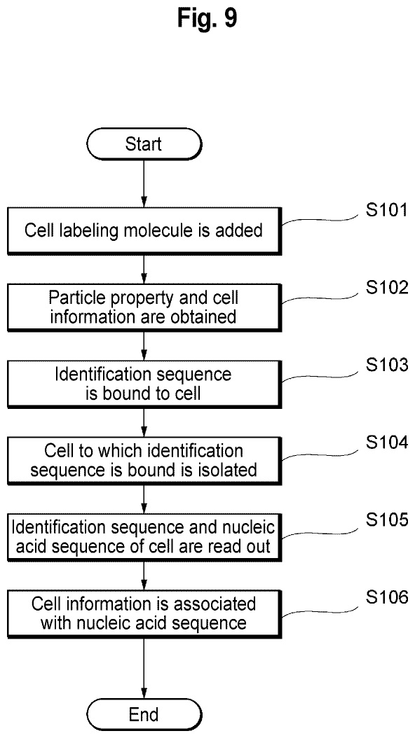

Next, in reference to FIG. 9, the method for analyzing a cell according to the embodiment will be described.

In Step S101, the cell labeling molecule according to the embodiment is added to a cell. For example, the cell labeling molecule is added to a medium containing the cell. The medium can be a liquid or a gel. A compartment including the cell and the cell labeling molecule can be formed from the medium including the cell and the cell labeling molecule. For example, when an aqueous medium including the cell and the cell labeling molecule is released from a pore to an oil, a water-in-oil emulsion is formed by phase separation, whereby a compartment including the cell and the cell labeling molecule can be formed. Alternatively, when an oil-based medium including the cell and the cell labeling molecule is released from a pore to an aqueous solution, an oil-in-water emulsion is formed, whereby a compartment including the cell and the cell labeling molecule is formed.

The number of cells included in each compartment can be one, or more than one. The number of cells included in each compartment can be adjusted by, for example, adjusting the concentration of cells in a medium containing cells before forming the compartment.

The number of the cell labeling molecules included in each compartment can be one, or more than one. The number of the cell labeling molecules included in each compartment can be adjusted by, for example, adjusting the concentration of cell labeling molecules in a medium containing cell labeling molecules before forming the compartment. Hereinbelow, an example in which the number of the cell labeling molecules included in each compartment is more than one will be described.

For example, when 50 particles having different properties from each other are used, and five particles are included in each compartment, 1,000,000 or more combinations of properties of the particles can be made. Accordingly, even when a large number of compartments are formed, the same combination of the properties of the particles in each compartment almost never be made. For this reason, each of a large number of compartments can be distinguished by the combination of the properties of the included particles.

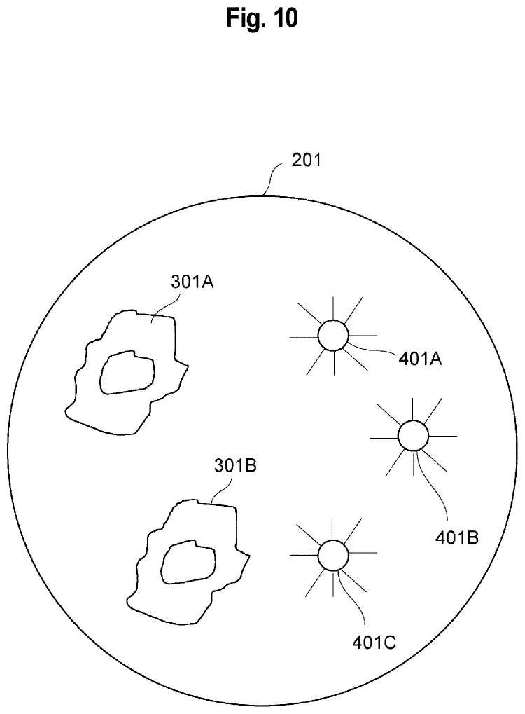

FIG. 10 schematically shows a compartment 201 in which two cells 301A, 301B and three cell labeling molecules 401A, 401B, 401C are included.

In Step S102, data including the property of the particle and the non-destructive information of the cells are obtained. The data can be optically obtained. For example, the property of the particle and the non-destructive information of the cells can be obtained using the same device. The property of the particle and the non-destructive information of the cells can be obtained by the same method. The property of the particle and the non-destructive information of the cells can be obtained simultaneously. In the data, the property of the particle and the non-destructive information of the cells present near the particle are linked. Accordingly, when the property of the particle is identified, the non-destructive information of the cells present near the identified particle can be obtained. For example, when a particle and a cell are included in a compartment, and the property of the particle is identified, the non-destructive information of the cell included in the same compartment with the identified particle can be obtained. The data may be image data. The image data may include at least any of a fluorescence image, a bright field image, a dark field image, a phase contrast image, a differential interference image, a phase image, a Raman micrograph, an absorption spectrum image, and an autofluorescence spectrum image. Further, the non-destructive information of the cell may be a two-dimensional image or a three-dimensional image, over time information, non-image information such as Raman intensity and spectrum, autofluorescence intensity and spectrum, and absorption intensity and spectrum, and non-optical information such as sound, temperature, heat, and mechanical properties.

The properties of the particle are as described above. The non-destructive information of a cell is the information obtainable without destroying the cell and, for example, morphological characteristics of a cell and optical properties of a cell are included, but not limited thereto. The optical property of a cell is not limited, and examples include the color of transmitted light of a cell, the wavelength of transmitted light of a cell, the transmitted light spectrum of a cell, the phase shift of transmitted light of a cell, the transmittance of a cell, the absorption spectrum of a cell, the absorptivity of a cell, the color of reflected light of a cell, the wavelength of reflected light of a cell, the reflected light spectrum of a cell, the phase shift of reflected light of a cell, the reflectance of a cell, the color of scattered light of a cell, the wavelength of scattered light of a cell, the scattered light spectrum of a cell, the phase shift of scattered light of a cell, the color of fluorescence emitted from a cell, the wavelength of fluorescence emitted from a cell, and the spectrum of fluorescence emitted from a cell, but not limited thereto. The wavelength bands of transmitted light, absorption, reflected light, and scattered light are not particularly limited, and may be a visible light or an infrared light. The scattered light may include the Raman scattered light.

In Step S103, the linker of the cell labeling molecule is cleaved. For example, when the linker includes an UV cleavage molecule, the linker is cleaved by irradiating the linker with UV. Such an irradiation releases the molecule including the identification sequence and the binding molecule from the particle, and the identification sequence binds to the cell through the binding molecule. More than one type of the identification sequence corresponding to the type of properties of the particles present in the compartment binds to the cell. When the cells and the cell labeling molecules are included in the compartment, and the linker is cleaved, the molecules including the identification sequence and the binding molecule are dispersed in the compartment, and the identification sequence binds to the cells in the compartment through the binding molecules.

When the linker of the cell labeling molecule is cleaved, the linker of a specific cell labeling molecule can be selectively cleaved. For example, a UV light can be irradiated near the cell to be analyzed to selectively cleave the linker of the specific cell labeling molecule. A specific range may be irradiated with a UV light using a lens, a specific range may be irradiated with a UV light using a micromirror array, or a specific range may be irradiated with a UV light using a photomask.

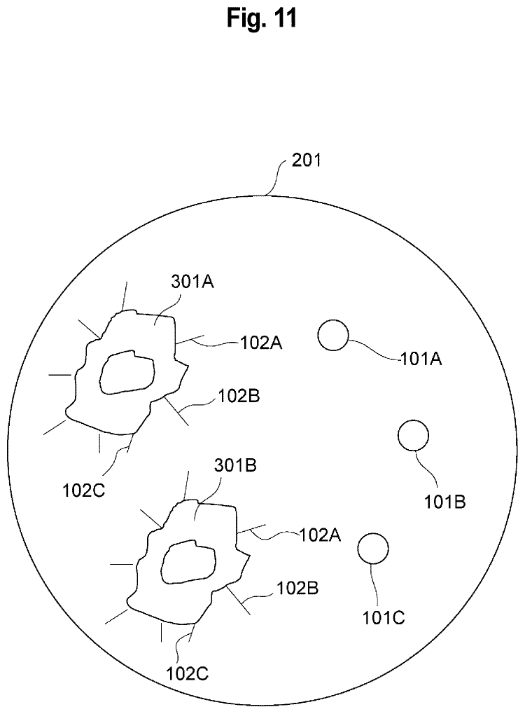

FIG. 11 schematically shows an example where, in the compartment 201, the identification sequences 102A, 102B, and 102C are released from the particles 101A, 101B, and 101C, and bind to the cells 301A, and 301B.

After Step S103, Step S102 can be carried out. Specifically, after the cleavage of the linker, the data including the properties of the particles and the non-destructive information of the cells may be obtained.

In Step S104, the cells to which the identification sequences are bound are isolated. The isolation method is not particularly limited. For example, the compartment is destroyed, and a population of cells to which the identification sequences are bound is obtained. When destroying the compartment, it is preferable for the cells not to be destroyed. Then, from the population, individual cells are isolated. For example, a single cell to which the identification sequences are bound can be dispensed to each of more than one well. Alternatively, a compartment including a single cell to which the identification sequences are bound can be formed. Alternatively, individual cells can be isolated by flow cytometry, individual cells can be isolated using a sequence labeling molecule such as an affinity tag. For example, to biotin bound to the identification sequence and/or the binding molecule, an avidin-modified magnetic bead can be bound, and the cell to which the identification sequences are bound can be isolated by a magnetic force.

When the cell is isolated, the particle may be removed. The particle can be removed by a centrifugal force or gravity. The particle can be removed using an optical tweezer. When the particle is magnetically charged, the particle can be removed by a magnetic force. A magnetic substance capable of specifically binding to the particle is bound to the particle, and the particle to which the magnetic substance is bound can be removed by a magnetic force. The particle can be chemically dissolved. For example, the particle can be enzymatically dissolved.

In Step S105, the nucleic acid sequence of the cell and the identification sequence bound to the cell are read out with respect to each isolated cell. For example, with respect to each isolated cell, the cell is dissolved and the nucleic acid derived from the cell is extracted. During this procedure, the more than one type of the identification sequences bound to the isolated cell is also extracted. When RNA is extracted, cDNA is generated from RNA using a reverse transcriptase. The reverse transcriptase can use DNA as a template. For this reason, when the identification sequence is DAN, the more than one type of the identification sequences bound to the isolated cell is also read out in the form of being included in cDNA. Then, by polymerase chain reaction (PCR), with respect to each isolated cell, the nucleic acid sequence of the cell and the more than one type of the identification sequences bound to the cell are amplified. Then, using a sequencer, with respect to each isolated cell, the nucleic acid sequence of the cell and the more than one type of the identification sequences bound to the cell are read out.

In Step S105, one or more than one non-destructive information of the cell and the nucleic acid sequence of the cell are associated. For example, each of the more than one type of the identification sequences read out with the nucleic acid sequence of the cell is in a unique relationship with the property of the particle before the linker is cleaved. Accordingly, it is possible to identify the combination of particles having the properties in a unique relationship with the combination of the read-out identification sequences. Thus, based on the combination of the read-out identification sequences, from the data obtained in Step S102, the corresponded particle combination can be identified. Further, the property of the particle is linked to the non-destructive information of the cell present near the particle. Accordingly, the non-destructive information of the cell in a unique relationship with the combination of the read-out identification sequences is identifiable. For example, when the non-destructive information of a cell is morphological characteristics, the nucleic acid sequence of the read-out cell and the morphological characteristics of the cell are associated. For example, when the nucleic acid sequence and the morphological characteristics of more than one cell are found correlated, such a nucleic acid sequence can be considered the cause of the morphological characteristics of the cell.

When more than one cell is included in one compartment, the nucleic acid sequences of the read-out cell can be determined to which of the more than one cell in the compartment corresponds, based on the known non-destructive information on the read-out nucleic acid sequences. For example, the nucleic acid sequence of the read-out cell can be determined to which of the more than one cell in the compartment corresponds, based on the known correlation of the known sequence included in the read-out nucleic acid sequence and the known characteristics of the non-destructive information of the cell.

For example, when more than one different types of cells are included in one compartment, the different types of cells are morphologically identifiable when obtaining the non-destructive information. Accordingly, based on whether the RNA of the read-out cell includes a sequence specific to the type of cell, the RNA of the read-out cell can be determined to which of the more than one different type of cells in the compartment corresponds.

For example, when a stem cell and a differentiated cell are included in one compartment, the stem cell and the differentiated cell are morphologically identifiable when obtaining the non-destructive information. Accordingly, based on whether the RNA of the read-out cell includes a sequence specific to the stem cell or includes a sequence specific to the differentiated cell, the RNA of the read-out cell can be determined to which of the stem cell or the differentiated cell in the compartment corresponds.

For example, in one compartment, more than one cell interacting with each other can be placed. According to the present embodiment, with regard to each of the more than one cell interacting with each other, the non-destructive information of the cells can be associated with the nucleic acid sequences of the cells.

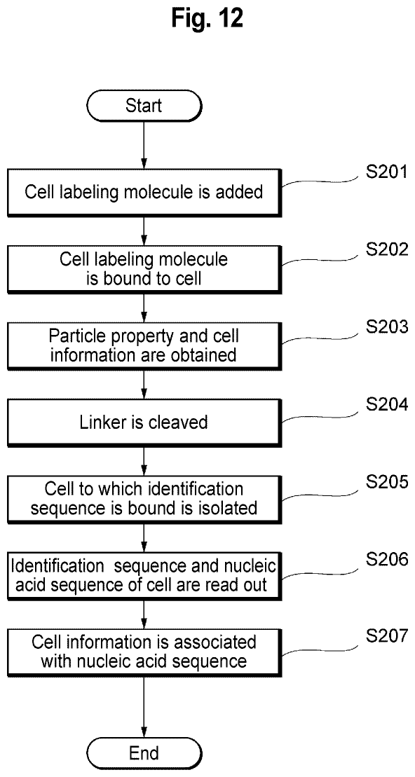

Next, in reference to FIG. 12, the method for analyzing a cell according to another embodiment will be described.

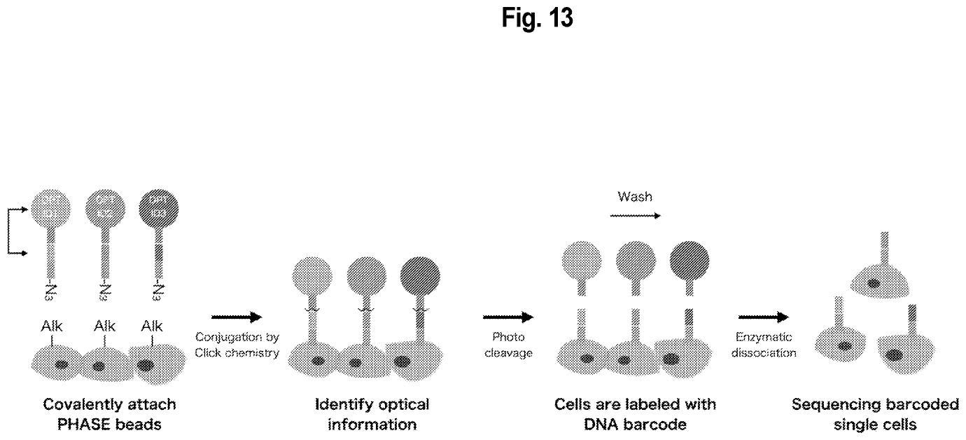

In Step S201, the cell labeling molecule according to the embodiment is added to cells. The cells herein, as shown in FIG. 13, may be adherent cultured. Alternatively, the cells may be at least a part of the tissue. For example, the cell labeling molecule is added to the culture solution in which the cells are cultured. In the method shown in FIG. 12, the cells do not need to be included in a compartment.

In Step S202, the cell labeling molecule sediments on the cell surface, and the cell labeling molecule binds to the cell through the binding molecule. The cell labeling molecule, for example, sediments on the cell surface by gravity. Alternatively, the cell labeling molecule may sediment on the cell surface by a centrifugal force. The cell labeling molecule can also be settled on the cell surface using an optical tweezer. When the particle of the cell labeling molecule is magnetically charged, the cell labeling molecule can also be settled on the cell surface by a magnetic force. A magnetic substance capable of specifically binding to the particle is bound to the particle of the cell labeling molecule, thereby settling the cell labeling molecule on the cell surface by a magnetic force.

In Step S203, as in Step S102 of FIG. 9, the data including the property of the particle and the non-destructive information of the cell is obtained. In Step S204 of FIG. 12, as in Step S103 of FIG. 9, the linker of the cell labeling molecule is cleaved. By this, the particle of the cell labeling molecule is released from the cell, and the identification sequence remains on the cell surface.

In Step S205 of FIG. 12, the cell to which the identification sequence is bound is isolated. The isolation method is not particularly limited. For example, when the cell to which the identification sequence is bound was adherent cultured, the cell is released from an incubator using a release agent or the like, and a population of cells to which the identification sequences are bound is obtained. When the cell to which the identification sequences are bound is at least a part of the tissue, the cell is dissociated from the tissue using a dissociative agent or the like, and a population of cells to which the identification sequences are bound is obtained. Then, as in Step S104 of FIG. 9, individual cells are isolated from the population.

In Step S206 of FIG. 12, as in Step S105 of FIG. 9, the nucleic acid sequence of the cell and the identification sequences bound to the cell by each isolated cell are read out. In Step S207 of FIG. 13, as in Step S105 of FIG. 9, one or more than one non-destructive information of the cell and the nucleic acid sequence of the cell are associated.

In Step S204 of FIG. 12, when the linker of the cell labeling molecule is cleaved, the linker of a specific cell labeling molecule can be selectively cleaved. In this instance, the cell to which the particle is bound where the linker was not cleaved can be removed. The cell to which the particle is bound can be removed by a centrifugal force or gravity. The cell to which the particle is bound can be removed using an optical tweezer. When the particle is magnetically charged, the cell to which the particle is bound can also be removed by a magnetic force. A magnetic substance capable of specifically binding to the particle is bound to the particle, the cell to which the particle to which the magnetic substance is bound is bound can also be removed by a magnetic force.

When the cell labeling molecule comprises an inhibitor that inhibits the function of the sequence labeling molecule, and the inhibitory function deactivates under the same conditions as the conditions that cleave the linker, when the linker of the cell labeling molecule binding to the cell is cleaved, the inhibitory function of the inhibitor deactivates, and the function of the sequence labeling molecule binding to the cell together with the sequence labeling molecule is demonstrated. Accordingly, using the sequence labeling molecule, the cell to which the sequence labeling molecule is bound can be isolated.

### Example 1 of the Embodiment: Production of Acrylamide Bead

A TBSET (Tris-Buffered Saline-EDTA-Triton) buffer solution containing 10 mmol/L Tris-HCl, 137 mmol/L NaCl, 2.7 mmol/L of KCl, 10 mmol/L EDTA, and 0.1% (v/v) Triton X-100 was prepared. Additionally, a 30% acrylamide/Bis solution (BIORAD, 29:1, #1610156), and an aqueous azo polymerization initiator (Wako, V-50, 2,2′-Azobis(2-methylpropionamidine) dihydrochloride, #017-21332) were prepared. The 10 hour half-life temperature (in water) of V-50 is 56° C.

A linker and a binding molecule-containing molecule were prepared, wherein, as the linker, a photocleavable spacer (IDT, iSpPC) that cleaves with UV light irradiation at 300 nm to 350 nm was inserted, as shown below. The linker-containing sequence has acrydite (Acryd) modified DNA at the 5′ terminal side. Acrydite reacts to acrylamide. The linker and the binding molecule-containing molecule have, on the 3′ terminal side, a sequence that functions as the binding molecule for binding to a cell. The linker is inserted between the acrydite modified DNA and the binding molecule.

/5Acryd/GGG/iSpPC/CCTTGGCACCCGAGAATTCCA

An acrylamide bead ingredient solution A, which contains a 6% (V/V) acrylamide/bis solution, a 1% (W/V) aqueous azo polymerization initiator, 10 μmol/L of acrydite-modified DNA in the 10% diluted TBSET buffer solution, was prepared.

The acrylamide bead ingredient solution A was suspended in an oil (BIORAD, Droplet Generation Oil for EvaGreen Assay, #1864112). Specifically, using an emulsification device (SPG, SPG micro kit, MG-20), the acrylamide bead ingredient solution A was extruded from a filter (SPG, SPG filter) having a pore diameter of 5 μm at a pressure of 8 to 9 kPa, thereby to prepare an emulsion. Alternatively, the acrylamide bead ingredient solution A and the oil were delivered to a microfluidic chip using a syringe pump (Harvard, PUMP 11 Elite, 70-4500), thereby to prepare an emulsion.

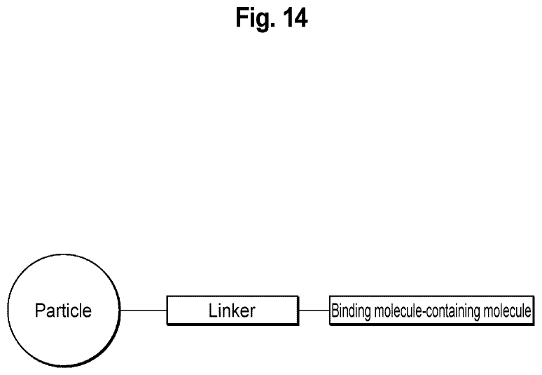

The emulsion was placed in a tube, and stirred at 56° C. for 2 hours using a rotary mixier under a nitrogen atmosphere, thereby to polymerize acrylamide. The TBSET buffer solution was layered on the emulsion, the oil layer was removed, and then Novec 7200 (3M, NOVEC 7200) containing 20 vol % 1H, 1H, 2H, 2H-Perfluoro-1-octanol (Wako, 324-90642) was added, thereby to extract the acrylamide bead to the aqueous layer. The oil remained in the aqueous layer at the bottom of the tube was washed with hexane (Wako, 085-00416) containing a non-ionic surfactant (Sigma, Span 80, 56635-250 ML), removed, and then the acrylamide bead was collected in a separate tube. By this, as schematically shown in FIG. 14, the acrylamide bead to which the linker and the binding molecule-containing molecule are bound was obtained.

### Example 2 of the Embodiment: Identification Sequence Modification on the Bead by Ligation

The 1^{st} identification sequence-containing molecule including the 1^{st} identification sequence as shown below was provided. The 1^{st} identification sequence-containing molecule had the 5′ terminal phosphorylated. The 5′ terminal sequence written in uppercase is the complementary sequence to the splint to be described later, and the sequence written in lowercase is the 1^{st} identification sequence. The 1^{st} identification sequence-containing molecule had the poly A sequence at the 3′ terminal.

pTTACCGACgtctactagtAAAAAAAAAAAAAAAAAAAAAAAAAAAAAA

The 2^{nd} identification sequence-containing molecule including the 2^{nd} identification sequence as shown below was provided. The 2^{nd} identification sequence-containing molecule had the 5′ terminal phosphorylated. The 5′ terminal sequence written in uppercase is the complementary sequence to the splint, and the sequence written in lowercase is the 2^{nd} identification sequence. The 2^{nd} identification sequence-containing molecule had the poly A sequence at the 3′ terminal.

pTTACCGACatcaggcctcAAAAAAAAAAAAAAAAAAAAAAAAAAAAAA

The splint as shown below was prepared. The splint had the sequence complementary to the 3′ terminal side sequence of the linker and the binding molecule-containing molecule of Example 1 and the sequence complementary to the 5′ terminal side sequence of the identification sequence-containing molecule.

GUCGGUAAUGGAAUU

A ligase (New England Biolabs, SplintR ligase), a ligase reaction buffer solution (New England Biolabs, 10× SplintR Ligase Reaction Buffer), and nuclease-free water (Qiagen, DNase/RNase-Free water, 129114) were prepared. Additionally, endoribonuclease (New England Biolabs, RNase H) and a ribonuclease reaction buffer solution (New England Biolabs, RNase H Reaction Buffer) were provided.

25 μL of the acrylamide beads prepared in Example 1, 7.5 μL of one of the 100 μmol/L identification sequence-containing molecule, 7.5 μL of the 100 μmol/L splint, and 7.5 μL of the ligase reaction buffer solution were mixed with nuclease-free water, thereby to prepare 70 μL of a substrate mixture. The substrate mixture was heated to 70° C. and cooled to room temperature at −0.1° C./sec.

5 μL of the ligase was added to the substrate mixture and incubated at 25° C. for one hour, whereby the linker and the binding molecule-containing molecule on the acrylamide bead and the identification sequence-containing molecule were bound. Subsequently, the acrylamide bead was washed with the PBST buffer solution.

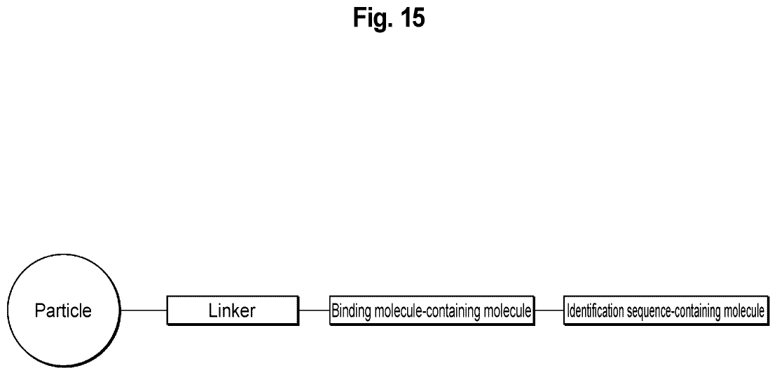

The acrylamide bead was suspended in 89 μL of nuclease-free water, 10 μL of a ribonuclease reaction buffer solution and 1 μL of endoribonuclease were added to the suspension and incubated at 37° C. for 20 minutes, thereby to break down the splint. Then, the acrylamide bead was washed with a TBSET buffer solution. By this, as schematically shown in FIG. 15, the acrylamide bead to which the linker, the binding molecule-containing molecule, and the identification sequence-containing molecule are bound was obtained.

### Example 3 of the Embodiment: Identification Sequence Modification on the Bead by Extension

A primer for the 1^{st} identification sequence as shown below was provided. The 3′ terminal side sequence of the primer for the 1^{st} identification sequence has the sequence complementary to the 3′ terminal side sequence of the linker and the binding molecule-containing molecule of Example 1. Using the poly T sequence and the sequence written in lowercase of the 5′ terminal side as the template, the extension reaction to be described layer is carried out.

TTTTTTTTTTTTTTTTTTTTTTTTTTTTTTactagtagacgtcggtaaT
GGAATTCTCGGGTGCCAAGG

A primer for the 2^{nd} identification sequence as shown below was provided. The 3′ terminal side sequence of the primer for the 2^{nd} identification sequence has the sequence complementary to the 3′ terminal side sequence of the linker and the binding molecule-containing molecule of Example 1. Using the poly T sequence and the sequence written in lowercase of the 5′ terminal side as the template, the extension reaction to be described layer is carried out.

TTTTTTTTTTTTTTTTTTTTTTTTTTTTTTtatcgttgcgaggtcactT
GGAATTCTCGGGTGCCAAGG

A primer for the 3^{rd} identification sequence as shown below was provided. The 3′ terminal side sequence of the primer for the 3^{rd} identification sequence has the sequence complementary to the 3′ terminal side sequence of the linker and the binding molecule-containing molecule of Example 1. Using the poly T sequence and the sequence written in lowercase of the 5′ terminal side as the template, the extension reaction to be described layer is carried out.

TTTTTTTTTTTTTTTTTTTTTTTTTTTTTTcaagtatcgcgaatccgaT
GGAATTCTCGGGTGCCAAGG

dNTP Mix (dNTP Mix (10 mM each), ThermoFisher Scientific, cat #R0192), an extension reaction buffer reaction (New England Biolabs, 10× NEBuffer 2), and a polymerase (New England Biolabs, DNA Polymerase I, Large (Klenow) Fragment) were prepared. DNA Polymerase I and Large (Klenow) Fragment have the polymerize activity and the 3′ to 5′ exonuclease activity, and the 5′ to 3′ exonuclease activity is deactivated.

25 μL of the acrylamide bead prepared in Example 1, 7.5 μL of one of the 100 μmol/L primer for the identification sequence, 7.5 μL of the 10 mmol/L dNTP mix, and 7.5 μL of the extension reaction buffer solution were mixed with nuclease-free water, thereby to prepare 70 μL of a substrate mixture. The substrate mixture was heated to 70° C. and cooled to room temperature at −0.1° C./sec.

5 μL of the polymerase was added to the substrate mixture and incubated at 37° C. for one hour, whereby the identification sequence-containing molecule binding to the linker and the binding molecule-containing molecule was extended. Specifically, the identification sequence complementary to the sequence written in lowercase of the primer for identification sequence and the poly A sequence are bound to the linker and the binding molecule-containing molecule. Subsequently, the acrylamide bead was washed with the TBSET buffer solution. Further, the acrylamide bead was placed in 50 mmol/L NaOH, incubated at room temperature for five minutes, the acrylamide bead was washed to remove the primer for the identification sequence. By this, as in Example 2, the acrylamide bead to which the single-stranded linker, the binding molecule-containing molecule, and the identification sequence-containing molecule are bound was obtained.

### Example 4 of the Embodiment: Production of Alginate Bead

50 mmol/L CaCl₂) and 50 mmol/L EDTA were added to water, thereby to prepare an EDTA-calcium buffer solution having pH of 7.2. 2% (w/v) sodium alginate and the EDTA-calcium buffer solution were mixed in equal amounts, thereby to prepare an alginate bead ingredient solution. Additionally, acetic acid was added to an oil (BIORAD, Droplet Generation Oil for EvaGreen Assay, #1864112) so that the final concentration was 0.1% (V/V), thereby to prepare an acetate oil mixture.

An alginate bead wash buffer solution A having pH of 7.5 and containing 10 mmol/L Tris-HCl, 137 mmol/L NaCl, 2.7 mmol/L KCl, 15 mmol/L/L CaCl₂), and 0.1% (v/v) Triton X-100 was prepared.

An alginate bead wash buffer solution B having pH of 7.5 and containing 10 mmol/L Tris-HCl, 137 mmol/L NaCl, 2.7 mmol/L KCl, and 1.8 mmol/L CaCl₂) was prepared.

An alginate bead wash buffer solution C having pH of 7.5 and containing 10 mmol/L Tris-HCl, 137 mmol/L NaCl, 2.7 mmol/L KCl, 1.8 mmol/L CaCl₂), and 0.1% (v/v) Tween-20 was prepared.

The alginate bead ingredient solution was suspended in the oil (BIORAD, Droplet Generation Oil for EvaGreen Assay, #1864112). Specifically, the alginate bead ingredient solution and the oil were delivered to a microfluidic chip (microfluidic ChipShop, Fluidic 947) through a pressure-based flow controller (FLPG plus 2.3 bar pressure pump, Fluigent, cat #FLPG005J), thereby to prepare an emulsion.

The acetate oil mixture in an equal amount to the oil amount of the emulsion was added to the emulsion in the tube, and the emulsion was stirred at room temperature for five minutes using a rotary mixer. The alginate bead wash buffer solution A was layered on the emulsion, the oil layer was removed, and then Novec7200 (3M, NOVEC7200) containing 20 vol % 1H, 1H, 2H, 2H-Perfluoro-1-octanol (Wako, 324-90642) was added, thereby to extract the alginate bead to the aqueous layer. The oil remained in the aqueous layer at the bottom of the tube was washed with hexane (Wako, 085-00416) containing a non-ionic surfactant (Sigma, Span80, 56635-250ML), removed, and then the alginate bead was collected in a separate tube.

### Example 5 of the Embodiment: Linker, Binding Molecule, and Identification Sequence Modification on the Bead

A MES-Ca buffer solution having pH of 5.5 and containing 100 mmol/L MES, 300 mmol/L NaCl, and 15 mmol/L CaCl₂) was prepared. Twenty mg of 1-ethyl-3-(3-dimethylaminopropyl) carbodiimide hydrochloride (EDC) was added to 50 μL of water, thereby to prepare an EDC solution. 6.0 mg of N-β-maleimidopropionic acid hydrazide (BMPH) was added to 50 μL of the MES-Ca buffer solution, thereby to prepare a BMPH solution.

A molecule containing a linker, a binding molecule, and an identification sequence was prepared, wherein a thiol group and a photocleavable spacer (IDT, iSpPC) were inserted at the 5′ terminal as shown below. The molecule containing the linker, the binding molecule, and the identification sequence had the poly A sequence at the 3′ terminal.

/5ThioMC6-
D/GGG/iSpPC/CCTTGGCACCCGAGAATTCCATTACCGACAAAAAAAA
AAAAAAAAAAAAAAAAAAAAAA

Approximately 2×10⁷ alginate beads prepared in Example 4 were suspended in 900 μL of the MES-Ca buffer solution. To the suspension, the EDC solution and the BMPH solution were added and stirred at room temperature for 80 minutes using a rotary mixer, thereby to introduce a maleimide group to the alginate beads. The alginate beads were washed twice with the MES-Ca buffer solution, and the alginate beads were suspended in the alginate bead wash buffer solution C.

The molecule containing the linker, the binding molecule, and the identification sequence was activated using a reducing agent (ThermoFisher, Bond-Breaker TCEP Solution), 100 μL of 5 μmol/L of the molecule containg the linker, the binding molecule, and the identification sequence was added to the suspension of alginate beads, incubated at room temperature for two hours, thereby binding the molecule containing the linker, the binding molecule, and the identification sequence to the alginate beads. Subsequently, the alginate beads were washed with the alginate bead wash buffer solution C.

### Example 6 of the Embodiment: Labeling of Identification Sequence on Beads

A labeling sequence labelled with Cy5 and having the sequence complementary to the 3′ terminal side poly A sequence on the beads produced in Examples 2, 3, and 5 as shown below was prepared.

/Cy5/TTTTTTTTTTTTTTTTTTTTTTTTV

1 μmol/L labeling sequence was added to each of the solutions containing the beads produced in Examples 2, 3, and 5 and incubated, thereby to label each of the identification sequences on the beads produced in Examples 2, 3, and 5 with Cy5.

### Example 7 of the Embodiment: Binding of the Binding Mediating Molecule to Cells

K562 Cells (JCRB0019) were prepared. Further, the 1^{st} binding mediating molecule having cholesterol TEG capable of binding to the cell membrane and the sequence complementary to the binding molecule on the bead at the 5′ terminal as shown below was prepared.

/5CholTEG/GTAACGATCCAGCTGTCACTTGGAATTCTCGGGTGCCAA
GG

The 2^{nd} binding mediating molecule having cholesterol TEG capable of binding to the cell membrane at the 3′ terminal side and the sequence complementary to the 1^{st} binding mediating molecule at the 3′ terminal side as shown below was prepared.

AGTGACAGCTGGATCGTTAC/3CholTEG/

The complex of the 1^{st} binding mediating molecule and the 2^{nd} binding mediating molecule was added to K562 cells, thereby to bind the complex of the 1^{st} binding mediating molecule and the 2^{nd} binding mediating molecule to the cell membrane of K562 cells.

### Example 8 of the Embodiment: Compartment Formation

A PBS buffer solution to which 0.1% BSA, 0.4 mmol/L EDTA, and a 0.1% cell membrane stabilizer (Thermo Fisher, Pluronic F68) were added was prepared as the solution for forming a compartment. To the solution for forming the compartment, 2.0×10⁶ cells/mL of K562 cells prepared in Example 7 and 1.1×10⁷ beads/mL of the beads prepared in Example 6 were added.

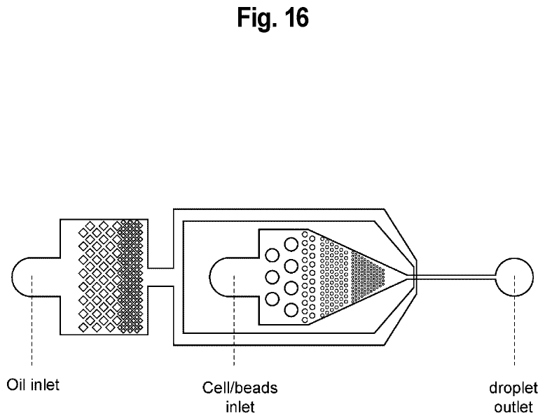

The solution for forming the compartment containing K562 cells and the beads and HFE7500 oil (3M, HFE7500 Novec 7500) containing 2% fluorosurfactant (008-fluorosurfactant, RAN Biotechnologies, RT008-N1G) were delivered to the microfluidic chip shown in FIG. 16 using a syringe pump (Harvard, PUMP 11 Elite, 70-4500), thereby to form a compartment having a diameter of about 90 μm, which was a droplet internally including K562 cells and the beads. The microfluidic chip was designed using an AutoCAD software (Autodesk). The delivery speed of the solution for forming a compartment was 7 μL/min. The delivery speed of the oil was 25 μL/min. Approximately four beads and approximately 0.5 cells were included per compartment. This means that the compartment that includes one cell was in a ratio of about 2 to 1.

### Example 9 of the Embodiment: Compartment Formation

The alginate bead wash buffer solution B was prepared as the solution for forming a compartment. To the solution for forming the compartment, 5.0×10⁵ cells/mL of K562 cells prepared in Example 7 and 1.1×10⁷ beads/mL of the beads prepared in Example 6 were added.

The solution for forming the compartment containing K562 cells and the beads and HFE7500 oil (3M, HFE7500 Novec 7500) containing 2% fluorosurfactant (008-fluorosurfactant, RAN Biotechnologies, RT008-N1G) were delivered to the microfluidic chip shown in FIG. 16 using a syringe pump (Harvard, PUMP 11 Elite, 70-4500), thereby to form a compartment having a diameter of about 85 μm, which was a droplet internally including K562 cells and the beads. The delivery speed of the solution for forming the compartment was 5 μL/min. The delivery speed of the oil was 25 μL/min. Approximately 3.5 beads on average and approximately 0.2 cells were included per compartment. This means that the compartment that includes one cell was in a ratio of about 5 to 1.

### Example 10 of the Embodiment: Linker Cleavage

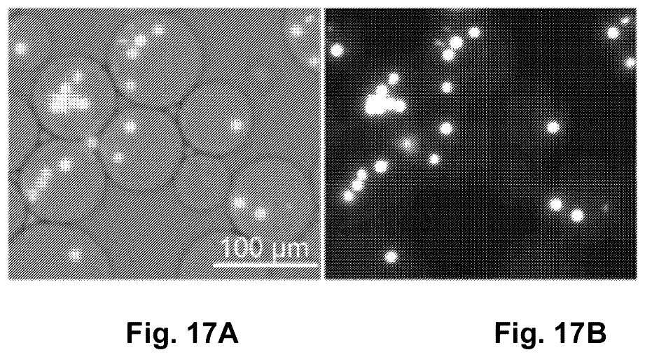

Using a microscope (Thermo Fisher, EVOS M7000), Cy5 for labeling the identification sequence included in the compartment prepared in Example 9 was excited, and the image observed in bright field is shown in FIG. 17A. The image observed in dark field is shown in FIG. 17B. It was confirmed that the identification sequences labelled with Cy5 were unevenly distributed on the beads in the compartment.

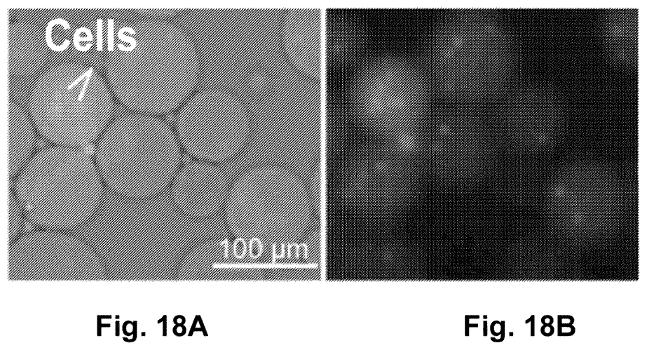

Then, the compartment was irradiated with a UV light for 10 seconds to cleave the linker, whereby the sequence including the identification sequence and the binding molecule was released from the bead. Immediately after the release, Cy5 was excited, and the image observed in bright field is shown in FIG. 18A. The image observed in dark field is shown in FIG. 18B. It was confirmed that the identification sequences labelled with Cy5 were released from the beads and dispersed in the compartment.

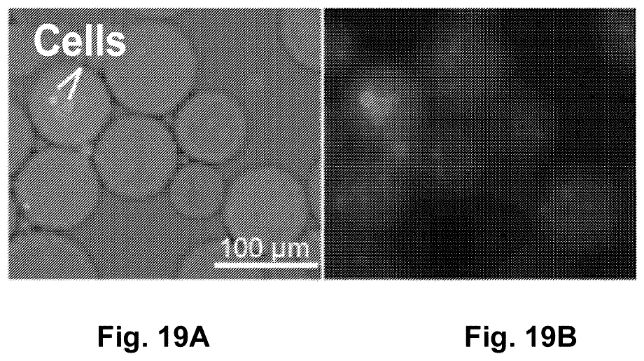

Seven minutes after the UV irradiation, Cy5 was excited, the image obtained by overlaying a Cy5 excited fluorescence micrograph image on a bright field image is shown in FIG. 19A. Additionally, FIG. 19B shows a Cy5 excited fluorescence micrograph image. It was confirmed that the identification sequences labelled with Cy5 were unevenly distributed on the cell membrane in the compartment. Thus, it was confirmed that the identification sequence bound to the cell membrane through the binding molecule.

### Example 11 of the Embodiment: Linker Cleavage

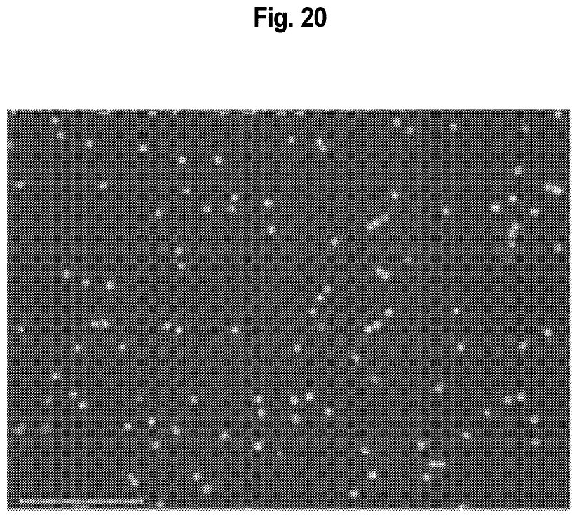

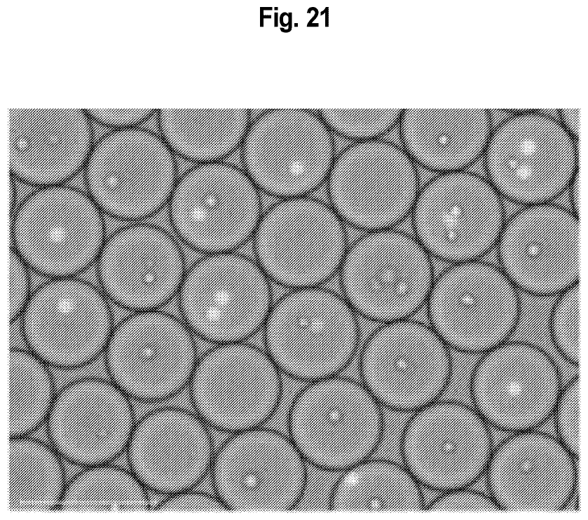

Cy5 was excited for the solution for forming the compartment containing K562 cells and the beads produced in Example 8, and the image observed in the dark field is shown in FIG. 20. Further, Cy5 was excited for the compartment produced in Example 8, and the image observed in the bright field is shown in FIG. 21.

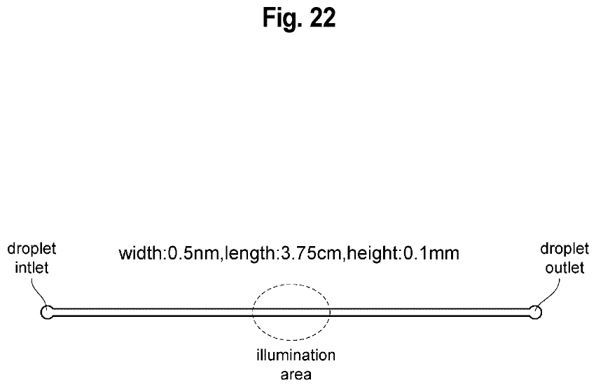

A polydimethylsiloxane (PDMS) chip having a straight flow channel having a length of 3.8 cm, a width of 500 μm, and a height of 100 μm as shown in FIG. 22 was prepared. To the part which is shown by surrounding with a dotted line and having a diameter of 500 μm at the center of the flow channel, the suspension of the compartment was passed through the flow channel at a flow rate of 5 μL/min using a syringe pump while irradiating a UV light using a microscope.

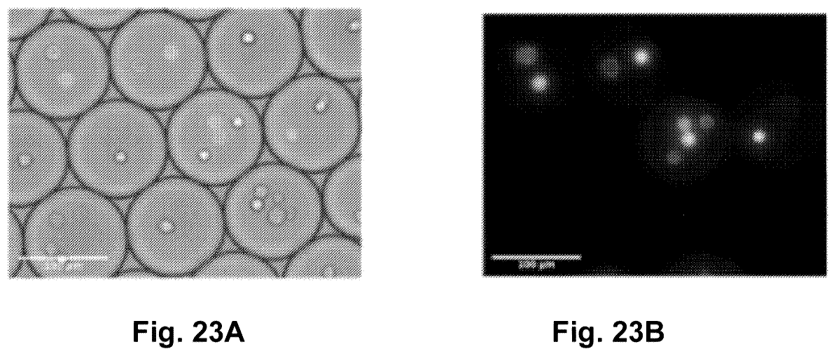

The suspension of the compartment passed through the flow channel was collected, and the image obtained by overlaying a Cy5 excited fluorescence micrograph image on a bright field image is shown FIG. 23A. Additionally, FIG. 23B shows an image observed by the Cy5 excited fluorescence micrograph image. It was confirmed that the identification sequences labelled with Cy5 were unevenly distributed on the cell membrane in the compartment. Thus, it was confirmed that the identification sequence bound to the cell membrane through the binding molecule.

### Reference Example 1 of the Embodiment: Linker Cleavage

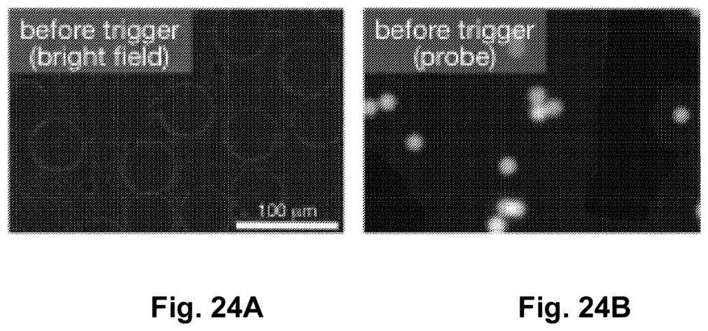

A compartment free of cells and including the beads to which the identification sequence labelled with Cy5 was bound through the linker was produced by the same method as in Example 8. The suspension of the compartment was placed in a petri dish, and the image observed in the bright field is shown in FIG. 24A. Cy5 is excited, and the image of the same area observed in the dark field is shown in FIG. 24B.

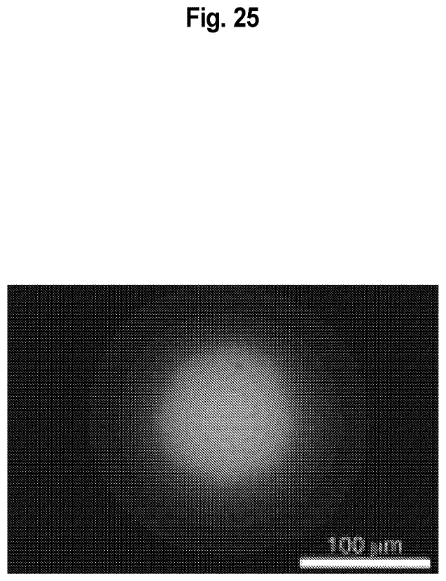

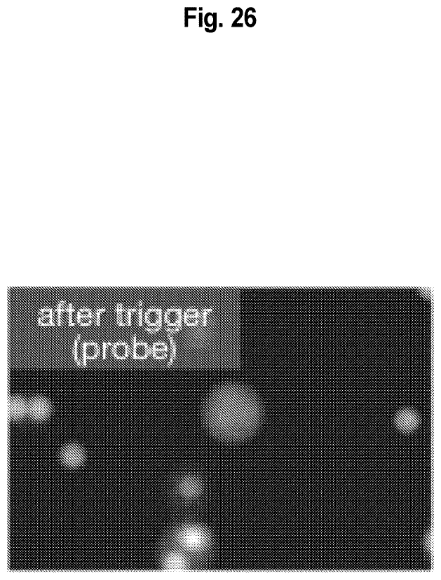

Next, as shown in FIG. 25, only to the compartments present at the center part of the image shown in FIGS. 24A-24B was irradiated with a UV light for five seconds, using a 40× objective lens of a microscope. Subsequently, Cy5 is excited, and the image of the same area observed in the dark field is shown in FIG. 26. It was confirmed that, in the compartments irradiated with the UV light, the identification sequences labelled with Cy5 were released from the beads and dispersed in the compartments, whereas in the compartments that were not irradiated with the UV light, the identification sequences labelled with Cy5 were not released from the beads and unevenly distributed on the beads.

### Reference Example 2 of the Embodiment: Linker Cleavage

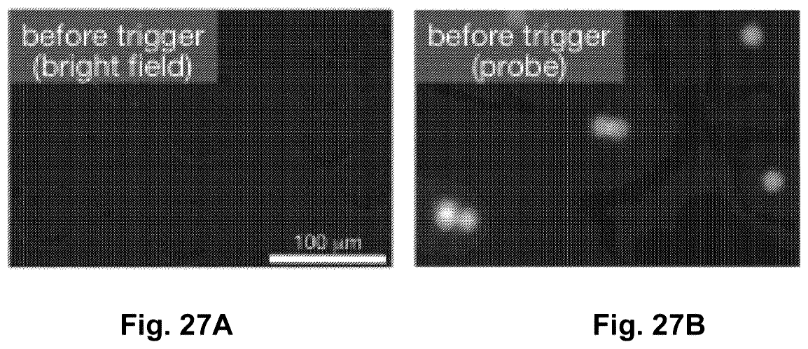

A compartment free of cells and including the beads to which the identification sequence labelled with Cy5 was bound through the linker was produced by the same method as in Example 8. The suspension of the compartment was placed in a petri dish, and the image observed in the bright field is shown in FIG. 27A. Cy5 is excited, and the image of the same area observed in the dark field is shown in FIG. 27B.

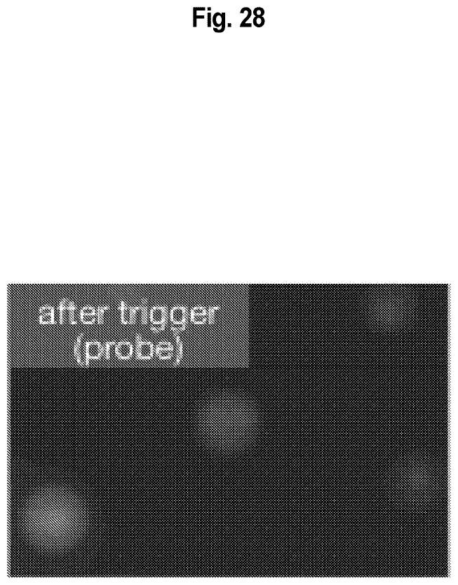

Next, all the compartments present in the image shown in FIGS. 27A-27B were irradiated with a UV light for five seconds. Subsequently, Cy5 is excited, and the image of the same area observed in the dark field is shown in FIG. 28. In all the compartments, the identification sequences labelled with Cy5 were released from the beads and dispersed in the compartments.

### Example 12 of the Embodiment: Enrichment

An acrylamide bead ingredient solution B was prepared, which contains a 6% (V/V) acrylamide/bis solution (BIORAD, 29:1, #1610156), a 1% (W/V) aqueous azo polymerization initiator (Wako, V-50, 2,2′-Azobis(2-methylpropionamidine) dihydrochloride, #017-21332), N-(3-BOC-aminopropyl) methacrylamide (Polysciences), and 10 μmol/L of acrydite-modified DNA as in Example 1 of the embodiment, in the 10% diluted TBSET buffer solution.

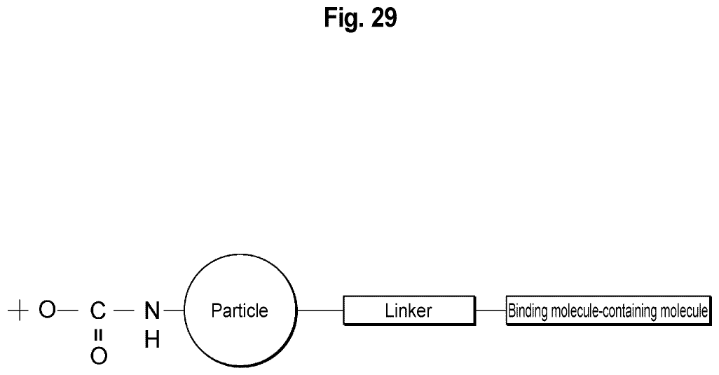

Using the acrylamide bead ingredient solution B, the acrylamide bead was produced in the same manner as in Example 1 of the embodiment. By this, as schematically shown in FIG. 29, the acrylamide bead to which the linker and the binding molecule-containing molecule, and a tert-butoxycarbonyl group (t-Boc) were bound was obtained.

The 3^{rd} identification sequence-containing molecule including the 1^{st} identification sequence as shown below was prepared. The 3^{rd} identification sequence-containing molecule had the 5′ terminal phosphorylated. The 5′ terminal sequence written in uppercase is the complementary sequence to the splint, and the sequence written in lowercase is the 1^{st} identification sequence. The 3^{rd} identification sequence-containing molecule had the poly A sequence and TEG modified with biotin at the 3′ terminal.

/5phos/TTACCGACgtctactagtAAAAAAAAAAAAAAAAAAAAAAAA
AAAAAA/3bioTEG

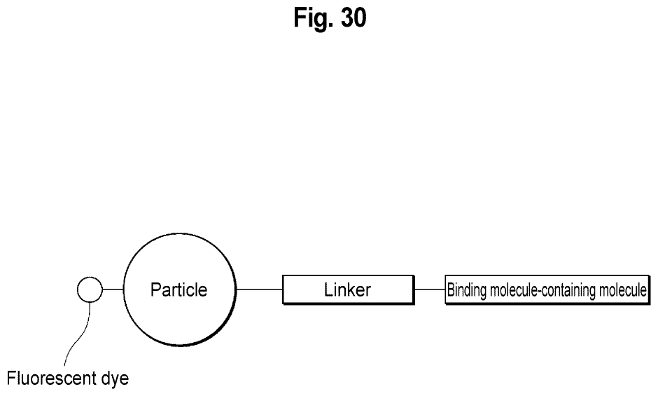

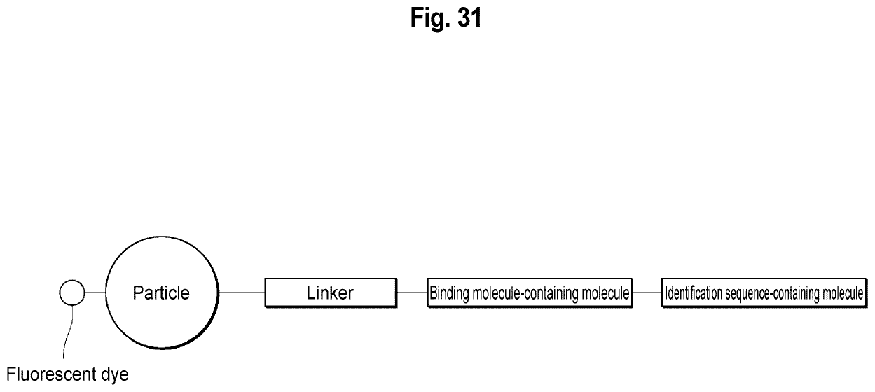

The amino group protected by t-Boc on the acrylamide bead surface was deprotected, and, as schematically shown in FIG. 30, the acrylamide bead surface was labelled with Cy5 (Cyanine5 NHS ester, Amine-reactive red emitting fluorescent dye, abcam). Then, using the 3^{rd} identification sequence-containing molecule, the linker and the binding molecule-containing molecule on the acrylamide bead were bound to the identification sequence-containing molecule by the same method as in Example 2 of the embodiment. By this, as schematically shown in FIG. 31, the Cy5-labelled acrylamide bead to which the linker, the binding molecule-containing molecule, and the identification sequence-containing molecule were bound was obtained.

K562 Cells were stained at room temperature for 30 minutes with a FITC labelled antibody (anti-CD71-FITC, BioLegend, 10 μL/2.5×10\hat{ }6 cells in 100 μL PBS) or an AF647 labelled antibody (anti-CD71-AF647, BioLegend, 10 μL/2.5×10\hat{ }6 cells in 100 μL PBS), and washed with PBS. Further, the complex of the 1^{st} binding mediating molecule and the 2^{nd} binding mediating molecule was introduced to the cell membrane of the FITC-labelled K562 cells and the AF647-labelled K562 cells, respectively, by the same method as in Example 7 of the embodiment.

A compartment was formed by the same method as in Example 8 of the embodiment, using 5×10⁶/mL of the FITC-labelled K562 cells and 3×10⁶/mL of the Cy5-labelled acrylamide beads. Additionally, as a control, a compartment that included the AF647-labelled K562 cells and was free of beads was formed. Subsequently, the compartment including the FITC-labelled K562 cells and the Cy5-labelled acrylamide beads and the compartment including the AF647-labelled K562 cells were mixed in a ratio of 1:1, thereby to obtain the mixture of the compartments.

The mixture of the compartments was irradiated with a UV light by the same method as in Example 11 of the embodiment, thereby to bind the identification sequence, the poly A sequence, and the biotin-modified TEG to the FITC-labelled K562 cells. Subsequently, PBS and then 20% PFO-HFE7500 oil were layered on the mixture of the compartments, thereby to break the compartments, and the suspension of the FITC-labelled K562 cells and the AF647-labelled K562 cells was collected.

Superparamagnetic beads modified with streptavidin (Streptavidin Microbeads, MACS) were added to the suspension of cells and incubated at 4° C. for 15 minutes. The cells were washed with PBS, and then the cells were resuspended in the buffer solution (MACS buffer: 0.5% BSA/2 mM-EDTA/PBS 0.5 mL), thereby to enrich the biotin-labelled cells using a MACS MS column. In the column, the cells to which the beads were bound were retained by the magnet, whereas the cells to which the beads were not bound flowed through the column and collected as the flow-through fraction. Subsequently, the magnet was removed from the column, the cells to which the beads were bound were eluted from the column, and the eluate was collected as the concentrated solution of cells to which the beads were bound.

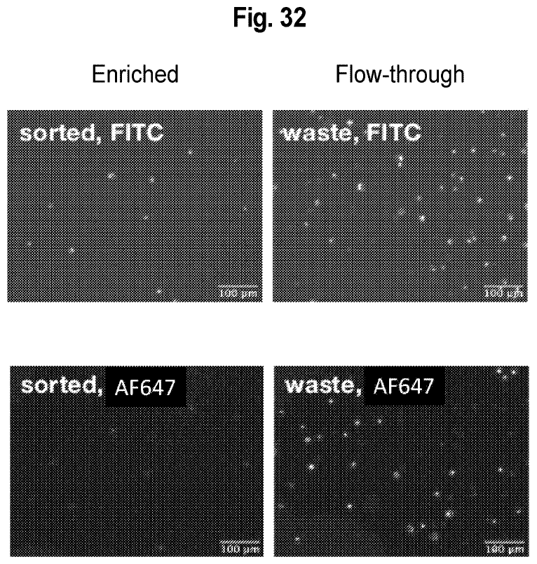

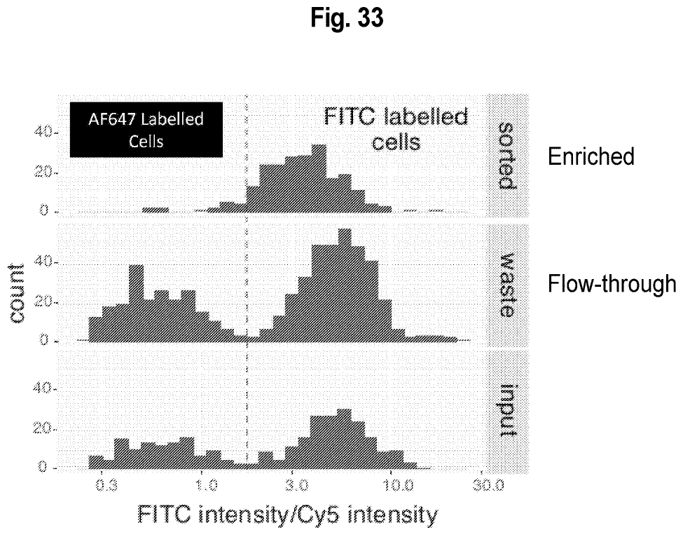

The suspension of cells input in the column, the flow-through fraction, and the concentrated solution of cells to which the beads were bound were photographed under the same condition. The images photographed were analyzed using an image processing software (Image J) and programming languages (Python3 and R language), only the cells were segmented, and then the total amount of the FITC labels and the total amount of the AF647 labels were calculated. The procedure referred to Van Rossum, G., & Drake, F. L. (2009). “Python 3 Reference Manual”, Scotts Valley, CA: CreateSpace; R Core Team (2020). “R: A language and environment for statistical computing”, R Foundation for Statistical Computing, Vienna, Austria. https://www.R-project.org/; Schindelin, J.; Arganda-Carreras, I. & Frise, E. et al. (2012), “Fiji: an open-source platform for biological-image analysis”, Nature methods 9 (7): 676-682, PMID 22743772. The results are shown in FIG. 32 and FIG. 33. It was indicated that the FITC-labelled K562 cells to which the identification sequences were bound could be separated from the AF647-labelled K562 cells to which the identification sequences were bound by the enrichment.

### Example 13 of the Embodiment: Sequencing

By the same method as in Examples 2 and 12 of the embodiment, the FITC-labelled acrylamide bead to which the linker, the binding molecule-containing molecule, and the 1^{st} identification sequence-containing molecule were bound, and the FITC-labelled acrylamide bead to which the linker, the binding molecule-containing molecule, and the 2^{nd} identification sequence-containing molecule were bound were prepared.

The complex of the 1^{st} binding mediating molecule and the 2^{nd} binding mediating molecule was introduced to the cell membrane of K562 cells by the same method as in Example 7 of the embodiment.

A 1^{st} compartment including the acrylamide bead to which the 1^{st} identification sequence-containing molecule was bound and K562 cells was formed by the same method as in Example 8 of the embodiment. Additionally, a 2^{nd} compartment including the acrylamide bead to which the 2^{nd} identification sequence-containing molecule was bound and K562 cells was formed. The 1^{st} compartment and the 2^{nd} compartment were mixed in a ratio of 1:1, thereby to obtain the mixture of the compartments.

The mixture of the compartments was irradiated with a UV light by the same method as in Example 11 of the embodiment, thereby to bind the 1^{st} identification sequences to K562 cells in the 1^{st} compartment, and to bind the 2^{nd} identification sequences to K562 cells in the 2^{nd} compartment. Subsequently, PBS and then 20% PFO-HFE7500 oil were layered on the mixture of the compartments, thereby to crush the 1^{st} and 2^{nd} compartments, and the suspension of K562 cells to which the 1^{st} identification sequences were bound, K562 cells to which the 2^{nd} identification sequences were bound, and the acrylamide beads were collected.

For removing the acrylamide beads from the suspension, superparamagnetic beads (Anti-FITC MicroBeads, Myltenyi Biotec, cat #130-048-701) bound to FITC binding to the acrylamide bead were added to the suspension and incubated at 4° C. for 15 minutes. Subsequently, the cells and the acrylamide beads were washed with PBS, and then the cells and the acrylamide beads were resuspended in the buffer solution (MACS buffer: 0.5% BSA/2 mM-EDTA/PBS 0.5 mL), thereby to remove the acrylamide beads using a MACS MS column. Using the fraction containing the cells that did not bind to the column, a single cell RNA library was generated.

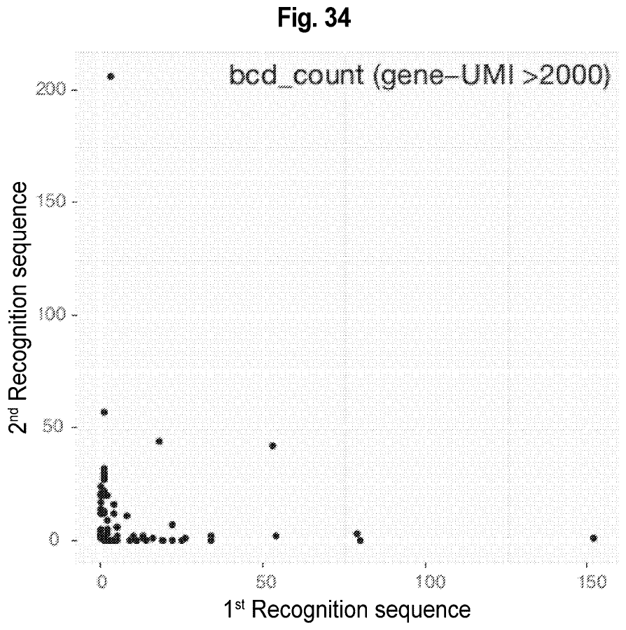

The generation of the single cell RNA library referred to Macosko, E. Z. et al, “Highly parallel genome-wide expression profiling of individual cells using nanoliter droplets,” Cell 2015, 161 (5), 1202-1214., doi: 10.1016/j.cell.2015.05.002. The fragment size of the library was analyzed using TapeStation 2200 (Agilent Technologies), and the library concentration was quantitatively determined by qPCR system CFX96 (BioRad) using a KAPA Library quantification universal kit (Roche). The sequencing was carried out in Illumina Mi-seq, using Mi-seq reagent v3 kit (Illumina). The sequencing read was analyzed using UMI-tools (see Smith, T., Heger, A. & Sudbery, I. UMI-tools: modeling sequencing errors in Unique Molecular Identifiers to improve quantification accuracy. Genome Res 27, 491-499, doi: 10.1101/gr.209601.116 (2017)), Cutadapt (see Martin, M. “Cutadapt Removes Adapter Sequences From High-Throughput Sequencing Reads” doi: https://doi.org/10.14806/ej.17.1.200 (2017)), BWA (see Li, H. & Durbin, R. “Fast and accurate short read alignment with Burrows-Wheeler transform” Bioinformatics 25, 1754-1760, doi: 10.1093/bioinformatics/btp324 (2009)), and SAMtools (see Li, H. et al. “The Sequence Alignment/Map format and SAMtools” Bioinformatics 25, 2078-2079, doi: 10.1093/bioinformatics/btp352 (2009)), and the R language was used for drawing. As shown in FIG. 34, the cells were positive to the 1^{st} identification sequence or the 2^{nd} identification sequence.

### Example 14 of the Embodiment: Enrichment

Magnetic bead having 2.5×10⁷ carboxyl groups (Micromer-M-COOH, micro-mod, cat #08-02-124) was washed twice with an MES buffer solution (MES: 100 mM, NaCl: 300 mM, pH 6.0). The beads were suspended in 500 μL of the MES buffer solution in which 38.4 mg (200 μmol) of EDC (N-(3-Dimethylaminopropyl)-N′-ethylcarbodiimide hydrochloride was dissolved. Next, to the suspension of magnetic beads, 10 μL of a solution containing 250 μmol/L of, as shown below, a linker and a binding molecule-containing molecule, wherein, as the linker, a photocleavable spacer (IDT, iSpPC) that cleaves with UV light irradiation at 300 nm to 350 nm was inserted, was added, and the suspension of the magnetic beads was incubated at room temperature for three hours while shaking the suspension of the magnetic beads, thereby to bind the linker and the binding molecule-containing molecule to the magnetic beads. The linker and the binding molecule-containing molecule had cholesterol TEG capable of binding to the cell membrane at the 5′ terminal. Further, the linker and the binding molecule-containing molecule had the amino group (AmMo) reactable to the carboxyl group of the magnetic bead at the 3′ terminal. Subsequently, the magnetic beads were washed four times with 50 mmol/L Tris buffer solution (pH 8.0) and suspended in PBS. For washing the magnetic beads, a magnetic stand (DynaMag™-2 magnet Thermo, cat #12321D) was used.

/5Chol-
TEG/GTAACGATCCAGCTGTCACTTGGAATTCTCGGGTGCCAAGG/
iSpPC/GGG/3AmMO/

Further, 7×10⁵ magnetic beads were suspended in 200 μL of a PBS solution containing 0.5 μmol/L of the identification sequence to which biotin was bound at the 3′ terminal as shown below. A part of the identification sequence below is complementary to the above linker and the binding molecule-containing molecule. The suspension was allowed to stand for five minutes at room temperature, thereby to bind the identification sequence to which biotin was bind at the 3′ terminal to the linker and the binding molecule-containing molecule on the magnetic beads. Subsequently, the magnetic beads were washed with a PBS solution containing 0.04% BSA. For washing the magnetic beads, a magnetic stand (DynaMag™-2 magnet Thermo #12321D) was used.

CCTTGGCACCCGAGAATTCCATCATTTAGAAAAAAAAAAAAAAAAAAAA
AAAAAAAAAA/3bio

K562 Cells stained with an anti-CD71 antibody labelled with Alexa Fluor (registered trademark) 647 and K562 cells stained with the anti-CD71 antibody labelled with FITC were prepared. Neither of the cells had the binding mediating molecule introduced.

A PBS buffer solution to which 0.4% BSA was added was prepared as the solution for forming a compartment. To the solution for forming the compartment, a suspension of 7.6×10⁶/mL of the magnetic beads and a suspension of 7.6×10⁶/mL of K562 cells labelled with AF647 were added. The solution for forming the compartment containing K562 cells and the magnetic beads and HFE7500 oil (3M, HFE7500 Novec 7500) containing 2% fluorosurfactant (008-fluorosurfactant, RAN Biotechnologies, RT008-N1G) were delivered to the microfluidic chip shown in FIG. 16 using a syringe pump (Harvard, PUMP 11 Elite, 70-4500), thereby to form a compartment having a diameter of about 100 μm, which was a droplet internally including K562 cells labelled with AF647 and the magnetic beads. The delivery pressure of the solution for forming a compartment was 120 mBar. The delivery speed of the oil was 40 μL/min. Subsequently, the compartment was irradiated with a UV light for 20 minutes to cleave the linker, whereby the sequence including biotin, the identification sequence and the binding molecule was released from the magnetic bead thereby to bind biotin and the identification sequence to K562 cells labelled with AF647.

As a control, to the solution for forming a compartment, magnetic beads and K562 cells labelled with FITC were added, thereby to form a compartment internally including K562 cells labelled with FITC and the magnetic beads. The compartment including K562 cells labelled with FITC and the magnetic beads was not irradiated with a UV light.

The compartment irradiated with a UV light and including the AF647-labelled K562 cells and the magnetic beads and the compartment free of the UV light irradiation and including the FITC-labelled K562 cells and the magnetic beads were mixed in a ratio of 1:1, thereby to produce the mixture. PBS and then 20% PFO-HFE7500 oil were layered on the mixture, thereby to break the compartments, and the cells were collected. The collected cells were washed with the PBS solution containing 0.04% BSA. Further, a small amount of cells was separated as the sample before sorting (input sample).

A solution (10 μL) of magnetic microbeads coated with streptavidin (Miltenyi Biotec) was added to the suspension of cells (3×10⁶ cells/mL to 5× 10⁶ cells/mL, 90 μL), and the suspension was incubated at 4° C. for 15 minutes, whereby the magnetic microbeads were bound to AF647-labelled K562 cells to which biotin and the identification sequence were bound. The cells were washed with PBS, and then the cells were suspended in the MACS buffer solution (0.5% BSA/2 mmol/L EDTA/PBS 0.5 mL), thereby to enrich K562 cells to which biotin and the identification sequence were bound using a MACS MS column (Miltenyi Biotec) and collect as the concentrated sample. The cells that did not bind to the MACS MS column and flowed therethrough were collected as the flow-through sample.

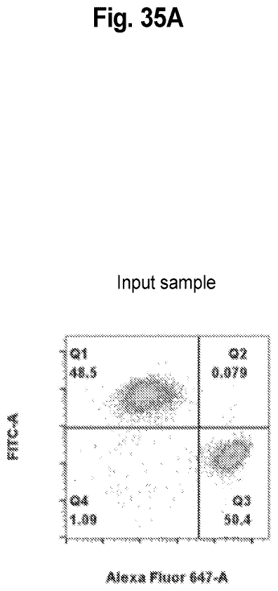

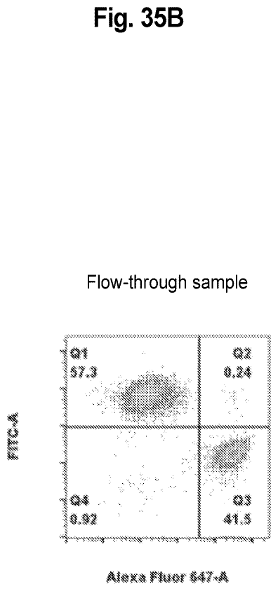

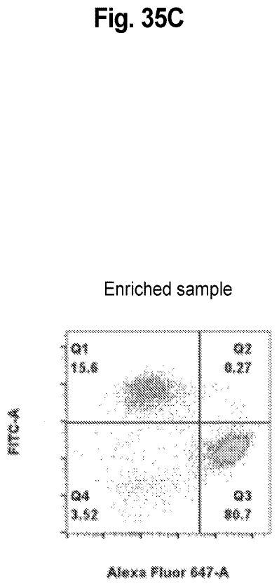

The input sample, the flow-through sample, and the enriched sample were respectively analyzed by FACS, and the total amount of the AF647 labels and the total amount of the FITC labels were calculated. For the analysis, FlowJo (registered trademark) software was used. As a result, as shown in FIG. 35A, in the input sample, the number of the FITC-labelled K562 cells and the AF647-labelled K562 cells were substantially the same. As shown in FIG. 35B, in the flow-through sample, the AF647-labelled K562 cells were reduced. As shown in FIG. 35C, in the enriched sample, the AF647-labelled K562 cells were enriched. Thus, it was indicated that MACS sorting can enrich K562 cells to which biotin and the identification sequence are bound.

### Example 15 of the Embodiment: Sequencing

A suspension containing the magnetic beads to which the linker and the binding molecule-containing molecule are bound as in Example 14 was prepared. Further, more than one PBS each containing DNA oligonucleotide (each concentration: 1 μmol/L) having 61 types of identification sequences different from each other was prepared. The sequences of 61 types of DNA nucleotides were as follows, and 8 mer Index sequence was different from each other. An example of the 8 mer Index sequence was TTACCGAC. The sequences of 61 types of DNA nucleotides all had the sequence complementary to the linker and the binding molecule-containing molecule on the magnetic beads. The suspension containing the magnetic beads and PBS containing DNA oligonucleotide having the identification sequence were mixed thereby to produce 61 types of the magnetic beads. The 61 types of the magnetic beads respectively had DNA oligonucleotides having identification sequences different from each other. The 61 types of the magnetic beads respectively washed with PBS, then the 61 types of the magnetic beads were mixed, and the mixed magnetic beads were washed with a PBS solution containing 0.04% BSA.

CCTTGGCACCCGAGAATTCCA[8 mer Index sequence]
AAAAAAAAAAAAAAAAAAAAAAAAAAAAAA

A PBS buffer solution to which 0.4% BSA was added was provided as the solution for forming a compartment. To the solution for forming a compartment, a suspension of 9.6×10⁶/ml of the mixed magnetic beads and a suspension of 7.6×10⁶/mL of K562 cells or THP-1 cells l were added. The solution for forming a compartment containing K562 cells or THP-1 cells and the magnetic beads and HFE7500 oil (3M, HFE7500 Novec 7500) containing 2% fluorosurfactant (008-fluorosurfactant, RAN Biotechnologies, RT008-N1G) were delivered to the microfluidic chip shown in FIG. 16 using a syringe pump (Harvard, PUMP 11 Elite, 70-4500), thereby to form a compartment having a diameter of about 100 μm, which was a droplet internally including K562 cells or THP-1 cells and 1 or more than one magnetic bead.

The compartment including K562 cells and one or more than one magnetic bead and the compartment including THP-1 cells and one or more than one magnetic bead were mixed in a ratio of 1:1, thereby to produce the mixture of the compartments. The mixture of the compartments was irradiated with a UV light for 20 minutes, thereby to bind one or more than one identification sequence to K562 cells and THP-1 cells. Subsequently, PBS and then 20% PFO-HFE7500 oil were layered on about 20000 compartments, thereby to break the compartments, and the cells were collected. The collected cells were washed with the PBS solution containing 0.04% BSA.

Of the collected cells, a single cell RNA sequencing library equivalent to about 10000 cells was generated using the 10× Chromium system (v3.1 kit). The system assigned the same cell barcode to RNA derived from the same single cell and to DNA having the identification sequence bound to the same single cell. Of the cDNA obtained during this process, fractions of short-chain DNA (<200 bp) that were not used for generating the single cell RNA sequencing library were collected using excess AMpure XP reagent, amplified using the following PCR primers 1 & 2, thereby to generate the sequencing library of the identification sequence for each cell. The fragment sizes of the obtained single cell RNA sequencing library and the sequencing library of the identification sequence were respectively analyzed using Tape Station 2200 (Agilent Technologies). Further, the library concentration was quantitatively determined by qPCR system CFX96 (BioRad) using KAPA Library quantification universal kit (Roche). Additionally, the sequencing of the library was carried out in a sequencer (Illumina Mi-seq), using Mi-seq reagent v3 kit (Illumina).

Primer 1
AATGATACGGCGACCACCGAGATCTACACGCCTGTCCGCGGAAGCAGTG
GTATCAACGCAGAGT*A*C
(the positions marked with * are phosphorothioate)
Primer 2
CAAGCAGAAGACGGCATACGAGAT[6 bases]
GTGACTGGAGTTCCTTGGCACCCGAGAATTCCA

The sequencing data were analyzed using UMI-tools (see Smith, T., Heger, A. & Sudbery, I. UMI-tools: modeling sequencing errors in Unique Molecular Identifiers to improve quantification accuracy. Genome Res 27, 491-499, doi: 10.1101/gr.209601.116 (2017)), Cutadapt (see Martin, M. “Cutadapt Removes Adapter Sequences From High-Throughput Sequencing Reads” doi: https://doi.org/10.14806/ej.17.1.200 (2017)), Cite-seq-Count (https://github.com/Hoohm/CITE-seq-Count), SAMtools (see Li, H. et al. “The Sequence Alignment/Map format and SAMtools” Bioinformatics 25, 2078-2079, doi: 10.1093/bioinformatics/btp352 (2009)), STAR (Dobin A. et. al. “STAR: ultrafast universal RNA-seq aligner” Bioinformatics. 2013; 29 (1): 15-21.), SCANPY (Wolf F. A. et. al. “SCANPY: large-scale single-cell gene expression data analysis” Genome Biol. 2018; 19 (1): 15.), and leiden (Traag. V. A. et. al. “From Louvain to Leiden: guaranteeing well-connected communities”, Sci Rep. 2019; 9 (1): 5233.). Further, the python language was used for drawing the analyzed data.

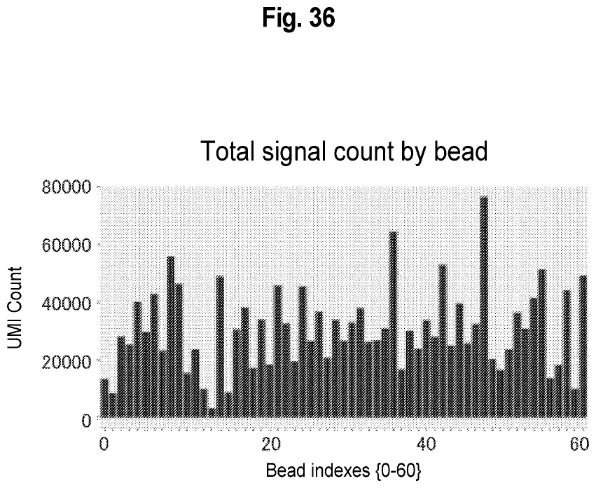

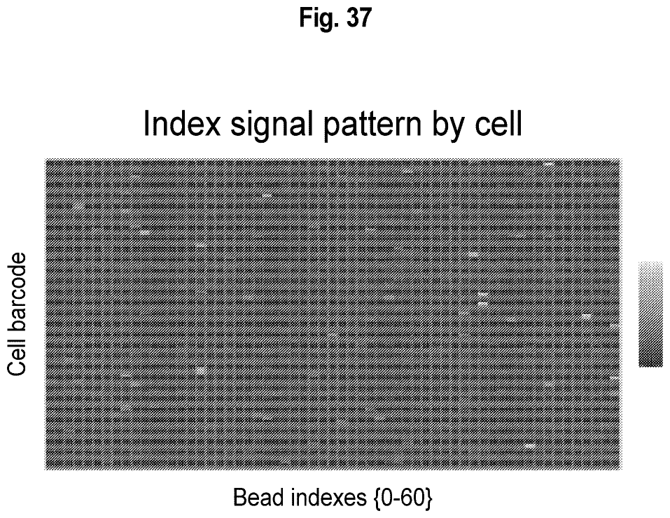

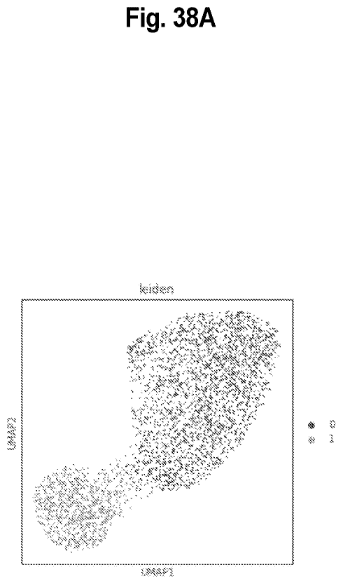

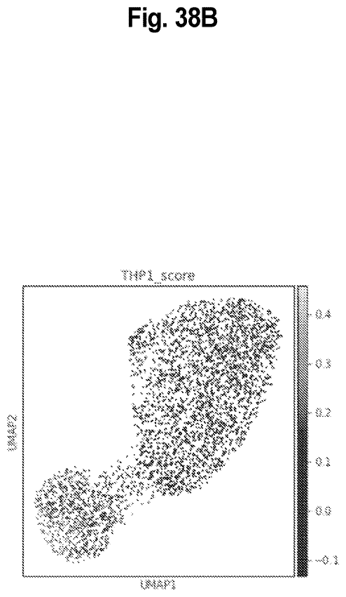

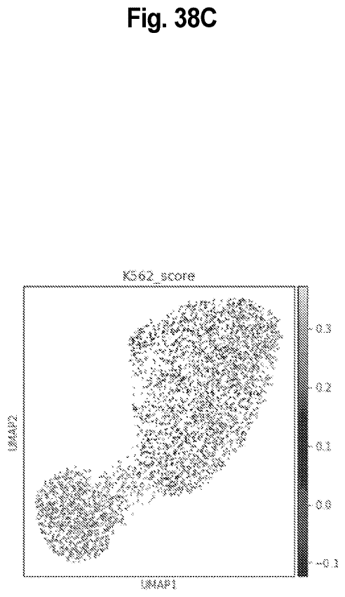

As a result, signals were obtained from 61 types of the identification sequences (bead indexes) as shown in FIG. 36. Additionally, as shown in FIG. 37, individual cells showed a signal pattern corresponding to at least one or at least any of the combinations of 61 types of the identification sequences. FIG. 37 shows an extraction of a part of the cells. In FIG. 37, the horizontal row corresponds to the single cell, showing that one or more than one identification sequence was bound to a single cell. Further, as shown in FIG. 38A, the cell data were clustered according to the expression patterns of single cell RNA using UMAP method and Leiden method, whereby the cell data were classified into two clusters. In FIG. 38A, the cluster lower left is classified as one, and the cluster upper right is classified as 0. In the original color drawing of FIG. 38B, the cluster lower left side of the two clusters was confirmed to have high scores on the RNA expression pattern distinctive to THP-1 cell and attributable to THP-1e cell. Additionally, in the original color drawing of FIG. 38C, the cluster upper right side of the two clusters was confirmed to have high scores on the RNA expression pattern distinctive to K562 cell and attributable to K562 cell.

### REFERENCE SIGNS LIST

101 . . . Particle, 102 . . . Identification sequence, 103 . . . Linker, 104 . . . Binding molecule, 105 . . . Poly A sequence, 106A . . . Sequence, 106B . . . Complementary sequence, 110 . . . Binding mediating molecule, 120 . . . Sequence labeling molecule, 130 . . . Inhibitor, 201 . . . Compartment, 301A . . . Cell, 401A . . . Cell labeling molecule

1. A cell labeling molecule, comprising:
a particle having an identifiable property,
an identification sequence that is identifiable and corresponds to the property of the particle,
a cleavable linker that binds the particle and the identification sequence, and
a binding molecule for binding to a cell, the biding molecule being bound to the identification sequence.

2. The cell labeling molecule according to claim 1, wherein the property of the particle bound to the identification sequence is capable of being specified based on the identification sequence.

3. The cell labeling molecule according to claim 1, wherein the particle is a bead.

4. The cell labeling molecule according to claim 1, wherein the identification sequence is a nucleic acid or an analog thereof.

5. The cell labeling molecule according to claim 1, wherein the identification sequence is a deoxyribonucleic acid or an analog thereof.

6. The cell labeling molecule according to claim 1, wherein the binding molecule is a molecule capable of binding to a molecule of a cell.

7. The cell labeling molecule according to claim 1, further comprising a sequence labeling molecule bound to the identification sequence and/or the binding molecule.

8. The cell labeling molecule according to claim 7, wherein the sequence labeling molecule includes a fluorescent molecule.

9. The cell labeling molecule according to claim 7, wherein the sequence labeling molecule includes an affinity tag.

10. A method for analyzing a cell, comprising:
adding, to at least one cell, a cell labeling molecule comprising a particle having an identifiable property, an identification sequence that is identifiable and corresponds to the property of the particle, a cleavable linker that binds the particle and the identification sequence, and a binding molecule for binding to a cell, the binding molecule being bound to the identification sequence and,
obtaining the property of the particle, and non-destructive information of the at least one cell,
cleaving the linker and binding the identification sequence to the at least one cell through the binding molecule,
isolating the at least one cell to which the identification sequence is bound, to read out the identification sequence and a nucleic acid sequence of the at least one cell, and
associating the non-destructive information of the at least one cell with the nucleic acid sequence of the at least one cell.

11. The method for analyzing a cell according to claim 10, further comprising:
placing the cell labeling molecule in a compartment, and
in the binding, binding the identification sequence to the at least one cell in the compartment.

12. The method for analyzing a cell according to claim 11, wherein the compartment is a gel or droplet.

13. The method for analyzing a cell according to claim 10, wherein the at least one cell is isolated by flow cytometry.

14. The method for analyzing a cell according to claim 10, wherein the at least one cell is isolated using an affinity tag.

15. The method for analyzing a cell according to claim 10,
wherein the cell labeling molecule further comprises a sequence labeling molecule bound to the identification sequence and/or the binding molecule, and
in the isolation, the sequence labeling molecule is used.

16. A method for analyzing a cell, comprising:
binding, to at least one cell, a cell labeling molecule comprising a particle having an identifiable property, an identification sequence that is identifiable and corresponds to the property of the particle, a cleavable linker that binds the particle and the identification sequence, and a binding molecule for binding to a cell, the binding molecule being bound to the identification sequence,
obtaining the property of the particle, and non-destructive information of the at least one cell,
cleaving the linker to release the particle from the cell,
isolating the at least one cell to which the identification sequence is bound, to read out the identification sequence and the nucleic acid sequence of the at least one cell, and
associating the non-destructive information of the at least one cell with the nucleic acid sequence of the at least one cell.

17. The method for analyzing a cell according to claim 16, wherein, when the cell labeling molecule is bound to the at least one cell, the at least one cell is adherent cultured.

18. The method for analyzing a cell according to claim 17, wherein, before isolating the at least one cell, the at least one cell is detached from an incubator.

19. The method for analyzing a cell according to claim 16, wherein, when the cell labeling molecule is bound to the at least one cell, the at least one cell is a part of a tissue.

20. The method for analyzing a cell according to claim 17, wherein, before isolating the at least one cell, the at least one cell is dissociated from the tissue.

21. The method for analyzing a cell according to claim 16, wherein the at least one cell is isolated by flow cytometry.

22. The method for analyzing a cell according to claim 16, wherein the at least one cell is isolated using an affinity tag.

23. The method for analyzing a cell according to claim 16, wherein the cell labeling molecule further comprises a sequence labeling molecule bound to the identification sequence and/or the binding molecule, and
in the isolation, the sequence labeling molecule is used.
